(19) United States
(12) Patent Application Publication
(10) Pub. No.: US 2015/0070319 A1
(43) Pub. Date: Mar. 12, 2015

(54) HUMAN INTERFACES FOR HOMES, MEDICAL DEVICES AND VEHICLES

(71) Applicant: Timothy R. Pryor, Sylvania, OH (US)

(72) Inventor: Timothy R. Pryor, Sylvania, OH (US)

(21) Appl. No.: 14/021,446

(22) Filed: Sep. 9, 2013

(51) Int. Cl. G06F 3/042; A61L 2/10; G06F 3/0488; G06F 3/0354; G06F 3/03

(52) U.S. Cl. CPC G06F 3/0425; G06F 3/03547; G06F 3/0308; G06F 3/04883; A61L 2/10; G06F 2203/04104; G06F 2203/04808
USPC 345/175

(57) ABSTRACT

Disclosed are novel method and apparatus for monitoring and aiding persons in vehicles and at home, especially senior citizens and others whose capabilities may be diminished. Electro-optical sensing of both person's body portions and physical controls is utilized, following from both disclosure on my RTD (Reconfigurable Tactile Display) invention as well as other gesture and motion based inventions in regard to sensing of control inputs from physical control (e.g. knobs, switches) positions and persons gestures. Applications to vehicles and the home are disclosed as well as applications to areas such as operating rooms and other locations in the hospital where sterility is paramount. A unique self-sterilizing control panel is also disclosed.

### CROSS-REFERENCE TO RELATED APPLICATIONS

The disclosures of the following patent applications are incorporated by reference in their entirety: U.S. provisional application 61/842,340, filed Jul. 2, 2013; U.S. patent application Ser. No. 12/748,666, filed Mar. 29, 2010; U.S. patent application Ser. No. 11/832,134, filed Aug. 1, 2007; U.S. provisional application 60/835,072, filed Aug. 3, 2006; U.S. patent application Ser. No. 11/349,350, filed Feb. 8, 2006; U.S. patent application Ser. No. 11/319,807 (now U.S. Pat. No. 7,671,851), filed Dec. 29, 2005; U.S. patent application Ser. No. 11/184,076 (now U.S. Pat. No. 7,466,843), filed Jul. 19, 2005; U.S. patent application Ser. No. 11/045,131, filed Jan. 31, 2005; U.S. patent application Ser. No. 10/934,762 (now U.S. Pat. No. 8,287,374), filed Sep. 7, 2004; PCT/US04/09701, filed Mar. 31, 2004; U.S. patent application Ser. No. 10/611,814 (now U.S. Pat. No. 7,489,303), filed Jul. 2, 2003; U.S. patent application Ser. No. 09/789,538 (now U.S. Pat. No. 7,084,859), filed Feb. 22, 2001; U.S. patent application Ser. No. 12/748,657, filed Mar. 29, 2010; U.S. patent application Ser. No. 12/748,665, filed Mar. 29, 2010; U.S. patent application Ser. No. 12/901,837, filed Oct. 11, 2010; U.S. patent application Ser. No. 12/793,165, filed Jun. 3, 2010; U.S. patent application Ser. No. 13/544,180, filed Jul. 9, 2012; U.S. patent application Ser. No. 13/613,679, filed Sep. 13, 2012; and U.S. patent application Ser. No. 13/668,351, filed Nov. 5, 3012.

### FIELD OF THE INVENTION

The invention is generally in the field of human computer interfaces, with application to medical instrumentation, instrument panels for vehicles, gestural and motion sensing applications and control and display devices in general. Disclosed are improvements and alternative embodiments of my RTD (Reconfigurable Tactile Display) invention disclosed in co-pending applications, as well as novel method and apparatus to allow operation in hospitals, nursing homes and other locations where sterility and cleanliness are paramount.

### BACKGROUND OF THE INVENTION

The invention addresses several human interface problems, particularly but not necessarily relating to activities in the home, in health care facilities and in vehicles. No known prior art discloses sensing of physical control locations using electro-optical sensing as I have disclosed in co-pending applications and have further disclosed here. In addition the disclosed invention is unique in that in some embodiments such sensing is combined with rear projection of data, while in others the sensing of both controls and persons is performed from a distance The most relevant background therefore for the present invention are the current conventional methods employed in cars, planes, hospitals, appliances and other devices.

With respect to a particular version of the invention having a self sterilizing feature useful in hospitals and other health care facilities, a recent patent application publication 2011/0256019 by Gruen et al filed Apr. 19, 2010, has disclosed a device for irradiating an LCD screen from behind to sterilize it. This device utilizes radiation from a large group of expensive UV LEDs to traverse the active display material which can cause degradation problems and warranty issues with such displays, in addition to adding considerable cost. And it discloses no means for dealing with physical controls which may be used in conjunction with a display.

### SUMMARY OF THE INVENTION

Disclosed are novel method and apparatus for monitoring and aiding persons in vehicles and at home, especially senior citizens and others whose capabilities may be diminished. Electro-optical sensing of both person's body portions and physical controls is utilized, following from both disclosure on my RTD (Reconfigurable Tactile Display) invention as well as other gesture and motion based inventions in regard to sensing of control inputs from physical control (eg knobs, switches) positions and persons motions or gestures. Applications to vehicles and the home are disclosed as well as applications to areas such as operating rooms and other locations in the hospital where sterility is paramount. In the latter case a unique self sterilizing control panel is disclosed.

Many of my co-pending disclosures have generally had some sort of rigid connection between the sensor/projector portion, and the screen/control surface portion. This application presents additional embodiments with either a completely separated projector/sensor from the screen/control surface, or embodiments where the two are not rigidly connected, but have an ability to swing, slide or otherwise move one in and out with respect to the other.

The invention also allows the separation of the electronic portion (projector and sensing of control and touch location) from the screen and control portion. This has numerous advantages in certain applications. For example the screen and control surface and controls attached thereto if any, can be simple, lightweight and easy to hold and clean and store or remove.

Further disclosed is apparatus which allows the use of conventional smart phone cameras and other cameras with retro-reflective targets on controls or persons or objects in a manner which is not disturbing to the person.

It is a goal of the invention to provide a self-sterilizing control panel and related systems for hospitals which overcomes previous problems and further provides many added features such as common device structures and physical controls as well as touch based controls.

It is another goal of the invention to provide a simplified common control system for persons to control TVs and provide optical motion detection for therapy, entertainment and other purposes

It is a goal of the invention to provide method and apparatus able to be easily removed, moved out of the way, or stored, which can provide control functions in appliances, vehicles, hospital beds, furniture, wheel chairs and other applications. It is further a goal to provide this in a safe manner which does not cause accidents or injury in the event of a crash.

It is a goal of the invention to provide method and apparatus which provides an easy to clean and maintain human interface comprising both display and controls, usable by patients or staff in hospitals, nursing homes, food preparation persons and other activities where sanitary conditions need to be maintained, while also requiring interaction with computers, and especially image based interaction.

It is a further goal to provide a screen and control surface that can be sterilized without damage to display or sensing electronics

It is a goal of the invention to provide method and apparatus that can provide minimum shock hazard to operators under a variety of adverse conditions including those where water or other liquids are present on the control panel itself.

It is a goal of the invention to provide method and apparatus for sensing touch locations and control detail state or position.

It is a goal of the invention to provide method and apparatus for providing projection on a freely held screen and control surface and sensing of controls or touch positions thereon.

It is a goal of the invention to provide method and apparatus for improving visibility of labels or other important information.

IT is a goal of the invention to provide means for using normal white light illumination sources provided on smart phones, tablets and the like for unobtrusive target detection for gesture detection and other control purposes

It is a goal of the invention to provide an oven whose window allows presentation of TV entertainment and act as a device for reconfigurable control functions.

It is a goal of the invention to provide method and apparatus, which can be resistant to damage by use of hard, flexible, or otherwise suitable materials for the screen/control surface.

It is an additional goal of the invention to provide means for maximizing the display space on car center stacks, digital camera backs, remote controls, and other objects where space is at a premium and must be shared with controls.

Further features and advantages of the present invention will be set forth in, or apparent from, the detailed description of preferred embodiments thereof which follows. In the disclosure herein, the term “projector/sensor unit” or “projector/sensor module” described here and in co-pending applications is meant to primarily denote a system combining a sensing and projection function, using flying spots or other arrangements such as DLP based projectors with associated camera systems. In addition, DLP projection systems run also in a sensing mode, can also form a projector/sensor unit and be used with the invention herein. Numerous types have been disclosed in the co pending applications incorporated by reference.

### BRIEF DESCRIPTION OF THE DRAWINGS

FIG. 1 illustrates a clean and sterilizable control panel for a hospital

FIG. 2a illustrates a self sterilizing control panel of the invention

FIG. 2 b illustrates self sterilizing knobs and switches

FIG. 2c illustrates a flip down control surface of the invention

FIG. 3 illustrates a camera based control system for assisting persons living at home or in another facility

FIG. 4 illustrates another embodiment of the invention in a hospital room

FIG. 5 illustrates further detail concerning the embodiment of FIG. 4

FIG. 6 illustrates camera sensing of gestures and controls with smart phones and other devices having LED flash

FIG. 7 illustrates further camera sensing aspects with tablets and smartphones

FIG. 8a illustrates a typical RTD control surface

FIG. 8b illustrates design of an exemplary knob

FIG. 8c illustrates another knob of the invention

FIG. 8e illustrates a whole surface dedicated to climate functions

FIG. 8f illustrates the ability of the invention to display very large images

FIG. 9 illustrates a center stack of a vehicle instrument panel of the invention in which the controls are color coded to enable easier operation or other advantages.

FIG. 10 illustrates another center stack embodiment in which three projectors in an over/under arrangement are used to maximize brightness and minimize depth;

FIG. 11 illustrates a basic sensor/projector embodiment also containing features for improving visibility of labels or other important information;

FIG. 12 illustrates an embodiment with screen/control surface in the steering wheel of a vehicle with the projector/sensor module in the steering column or instrument panel;

FIG. 13 illustrates a front projected sun visor based embodiment of the invention, wherein the projector/sensor unit is roof mounted;

FIG. 14 illustrates an embodiment of the invention in which a screen/control surface pulled down in front of the rear view minor of a vehicle;

FIG. 15a compares a RTD Automobile Instrument panel of the invention disclosed herein and in co-pending applications to that of a typical mid line or low line vehicle

FIG. 15b shows the Audio section of the instant invention

FIG. 15 c shows the RTD Audio section reconfigured to HVAC (climate)

FIG. 15d illustrates a blank control and display section 1500 with fixed physical controls in place

FIG. 15e illustrates the provision of switches to activate different functions

FIG. 15f illustrates radio (audio) function with heater (Climate) in virtual form.

FIG. 15g illustrates knob function of the invention

FIG. 16 illustrates systems for assisting persons in the kitchen

FIG. 17 illustrates additional kitchen systems

FIG. 18a is illustrates sensing of characteristics or actions persons in the kitchen

FIG. 18b is an embodiment facing a refrigerator in front view

FIG. 18c is another refrigerator embodiment

FIG. 19 illustrates an embodiment employing a front projection display

FIG. 20a illustrates a rear projection center stack with a physical control through it

FIG. 20b illustrates center stack display of right side camera blind spot images

FIG. 21 illustrates a portable embodiment of the invention for use as a keyboard;

FIG. 22 illustrates another portable embodiment used as a remote control

FIG. 23 illustrates a projector based control panel of the invention

FIG. 24 illustrates a vehicle center stack arrangement

FIG. 25a illustrates a low cost control panel arrangement of the invention

FIG. 25b illustrates a method of providing illuminated labels

FIG. 26 illustrates are switches according to the invention with center image data

FIG. 27 illustrates further switches according to the invention

FIG. 28 illustrates an appliance control panel of the invention

FIG. 29a illustrates a center stack of the invention in audio system mode;

FIG. 29b illustrates a center stack of the invention in climate control system mode;

FIG. 29c illustrates an embodiment of the invention for providing a detent or stop

### DESCRIPTION OF THE PREFERRED EMBODIMENTS

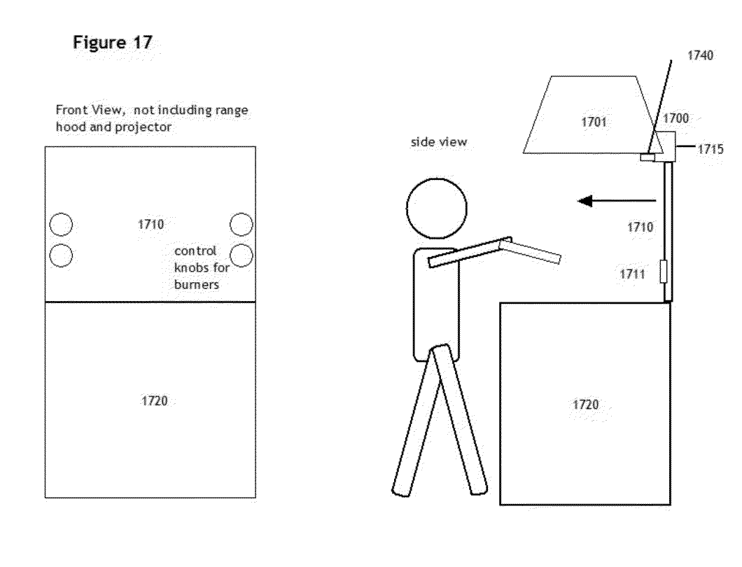

My application Ser. No. 12/748,666, of which this application is a continuation-in-part, discloses in FIG. 17 thereof a new form of easily sterilizable and cleanable control device for use in hospitals and nursing homes. The complete separation of the items contacted by persons from the operating electronics allows knobs and other physical controls and the touch screen to be easily cleaned or sterilized without harming the electronics or voiding the warranty thereof.

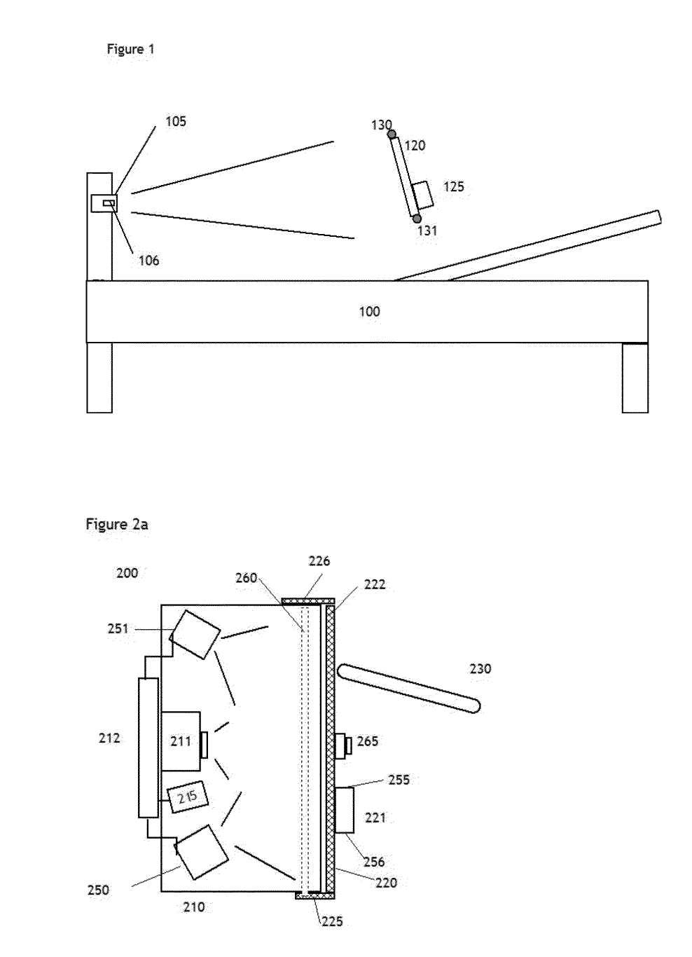

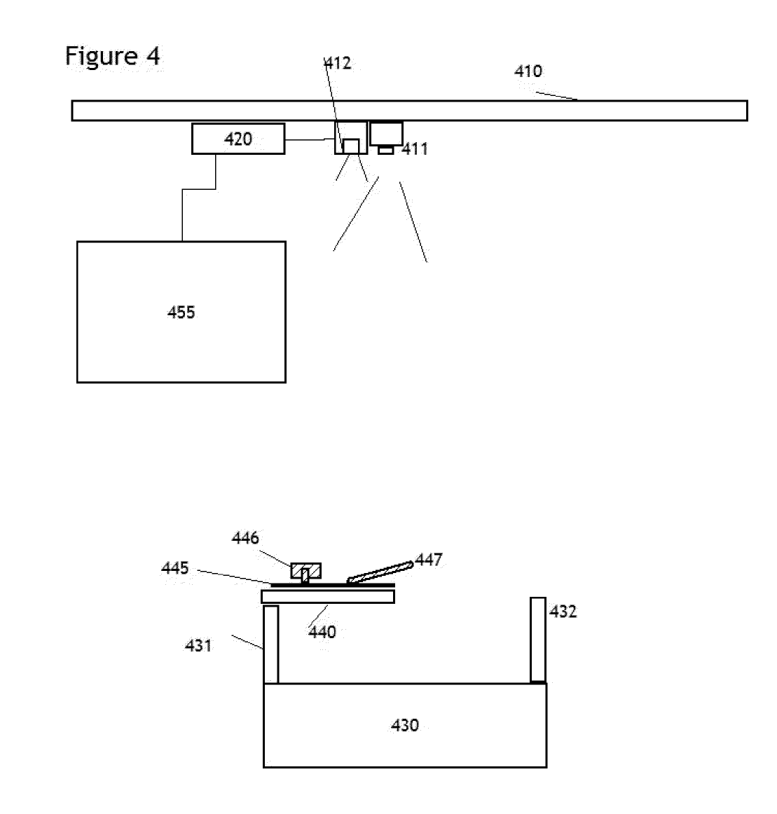

FIG. 1 illustrates a similar embodiment which makes use of the unique ability of the invention to have an easy to clean and even sterilize-able screen and control surface 120, in this case for use in a hospital bed 100. As shown the projector & sensor module 105 which includes camera 106 if a separate camera is required, is located at the foot of the bed, or alternatively in other locations such as the side of the bed, and illuminates and views the screen and control surface from the rear. A computer not shown, controls both projection and sensing, and can be interfaced to external sources such as the internet or cable providers. The Screen/control surface can be attached to the bed or freely held as shown here, where the patient laying in the bed 100 holds it on their lap. The patient himself is not shown here, for clarity. Control panels can be pulled out, or folded out from a side of bed as well. The person can not only see a projected TV program displayed on the screen, he can control the program with knobs 125 and/or other physical controls or touch functions which are sensed electro-optically as disclosed in co-pending applications incorporated by reference herein. The patient may use the device for selecting meals for the day, interacting with patient databases, sending emails, contacting the nurse's station, controlling lights and so forth. Individual control elements such as knobs and switches can themselves be attached to the bed (or other item such as a chair), as shown for example in front viewing example of FIG. 4.

It is important to note that in this example the Screen/control surface is lightweight (having no electronics, batteries or power requirements) inexpensive, is largely unbreakable, and has no wires or other problem items. The projector/sensor unit is out of the way and doesn't need to be moved. All electrical connections are between the projector and computer/Display/speakers, without causing a shock hazard or other problem for the patient handling the Screen/control surface remotely positioned from the electronics. This surface indeed has many advantages in this application. It is easy to move out of the way. It is light, thin, no wires, and simple plastic screen/control surface and thus is good for hospital beds, wheel chairs, and chairs in nursing homes or family rooms, and other applications. It is also removable and easy to store. The screen/control surface in some cases can be of flexible plastic and rolled up if such is desired.

In addition, where desired, the screen and control surface can be constructed of material that can be sterilized, either by heat or by radiation. This is a major advantage of the invention for application in hospitals and other health care facilities. It is noted that in this hospital bed application, or alternatively in a vehicle, or easy chair in one's home, or other location, the simple rear projection screen and control surface may be freely held with respect to the projector/sensor module using the ability of the system to realign its projection and sensing coordinates based on determination of the location of fixed datum's on the screen and control surface 120. The datum targets 130, 131 and generally others on the other two corners of the screen and control surface allow the sensor unit in 105 to determine the location of the screen and control surface 120 and adjust the projection and sensing image data positions accordingly, when the surface 120 is within a desired operating range. This is a dynamic function, which allows the person to move the screen and still get accurate projection and sensing within a certain accommodation range.

This capability also allows the system to sense using IR (for example using infrared LED illumination at 880 nm, and a camera responsive substantially to only this wavelength using a bandpass filter in an unobtrusive manner), and only turn the on the projector when the screen and control surface is in the accommodation range in both xyz and angular position. All 6 degrees of freedom of 120 with respect to projection and sensing 105 can be solved using data from the four datum's in known position on the corners of the screen and control surface in place.

The embodiment of FIG. 1 has illustrated what I believe is a novel and useful embodiment for persons in bed or other locations such as car seats where it may be desirable to have no physical connection between the screen/control surface and the projector and sensor unit (and associated computer, communication devices and the like). The bed application can be it in ones normal bed at home or confined to a hospital bed—the example illustrated and particularly germane given the problem of cleaning devices which patents hold in their hands such as TV remote control units. As pointed out a very big advantage of the invention is that no electronics are involved and control surfaces can be easily washed. The same approach can be used in beds in a home or hotel.

The goal is to provide both useful information and amusement to the patient, who as shown in FIG. 1 is able to hold a screen and control surface in his hand. This as discussed can be held in free space without a support since it is generally simply plastic and can be quite light. Alternatively, of course there can be brackets or other means used to sturdy the screen. Side rails or posts of the bed can be used to clamp the screen bracket to for example. The system also has an advantage that no wires or wireless transmission need connect the device in the patients hand, thus making it safer to use around sophisticated instrumentation such as heart monitors.

In other applications I've pointed out one can photogrammetrically determine position and orientation of the screen in free space using a sensing function built into the projector or a separate sensor such as a camera if used, typically located in this invention in the same housing as the projector.

The targets such as 130, 131 etc are viewed by the sensing system of the projector. Dynamically this data may be taken into the computer and utilized to correct the projection of information onto the screen so as to account for angular rotation and movement in and out by the patient of the screen toward the projector along the optical axis of the projector. For example, the image can either be caused to rotate with the screen to stay lined up on the screen or it can stay stationary and simply utilize the information gathered from the screen to cause the projector not to project past the edge of the screen if desired so as to not project into the eyes of the user for example. In this case it means that some of the image would be cut off, if the screen were rotated. The potential of z-axis movement means that the magnification of the image on the screen can vary. Conversely, one can choose to keep the is area of the projected image constant on the screen, which means that as one moves in and out certain areas would either be lost around the edges or conversely be a have a dead space around the edges.

The sensor unit sensing the position of the screen also performs another function, and that is, to determine the location of the patient's fingers or objects held by the patient such as a pen on the screen/control surface or certain controls on the screen and control surface such as knob 125. In this case the measurements taken are taken at relative to the screen and control surface through the use of the target datum's on the knob and the screen. These measurements in turn need to be corrected. However, if the image projection is being caused it to rotate or otherwise move on the screen, the knowledge of where a correct touch for example, corresponds to the actual projected image needs to be obtained by the computer.

The invention is extremely useful for hospital application because of the lightweight easy to clean interface for the patient. The users desires are optically transmitted to the computer of the device (in this case shown in the foot of the bed), which can then communicate over wires if necessary to any other device such as central computer of the hospital, a TV set on a wall, or whatever. And TV signals can be directed to the computer controlling the projector of the device in the bed in a similar manner. There really is no need for a wall mounted TV at all. Sound can be provided to the user from a small amplifier and speaker located on bed side, which also be provided with earphones if needed to avoid disturbing other patients.

Expanding on the above concept, another co-pending application Ser. No. 12/793,165 entitled “sterile, cleanable and reconfigurable human interfaces” has disclosed apparatus for use by surgeons in operating rooms, and other hospital applications. Once again, the lack of connection between the sensing and illumination on the one hand, and the control surfaces and touch display on the other, allows sterility to be achieved at low cost. And the ability to easily interchange the screen and controls allows one to easily substitute a clean panel for a contaminated one.

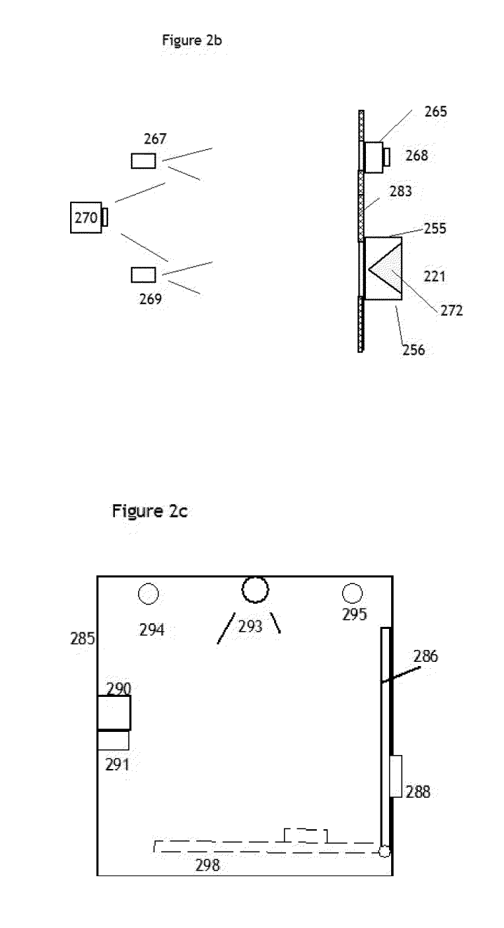

It is also possible to have a touch screen control panel according to the invention which can sterilize itself, adding to convenience and assuring that sterile conditions are maintained without relying on human activity. Illustrated in FIG. 2 herein is an improved self sterilizing control panel along the lines of FIG. 12 of above referenced Ser. No. 12/793,165 application and other co-pending referenced applications.

FIG. 2a illustrates a control panel 200 comprised of a housing 210 in which are contained a projector 211 for projecting image information on a rear projection screen and control surface 220. The projector is controlled by computer 212 which also receives inputs from various touch and physical controls in order to execute commands of a user of the control surface. The surface 220 may also have physical control details such as knob 221 and push button switch 270 A push switch function may also be incorporated in to the knob as shown in co-pending applications. Using camera 215 the surface may act as a touch screen as to be touched by one or more fingers of a user 230 in order to register commands responsive to information projected on the surface from the rear thereof. The camera can also be used to sense the position or state of the physical controls such as knob and switch shown

As taught in the previous applications the screen and control surface 220 including any physical controls mounted thereto may be sterilized elsewhere and brought in a clean sterile state to be affixed to the housing with sterile side shields such as 225 and 226 fitting over the housing such that the unclean housing is not touchable by the user in normal operation. (It is noted that the side shields would typically be on all sides, the two sides in and out of the plane of the drawing being not shown here for clarity). When an operation or other procedure is finished the now contaminated screen and control surface 220 is taken off and interchanged with a new sterile one for the next operation.

In cases where one cannot interchange the screen and control surface, it is desirable to have a self-sterilizing capability as provided herein. Radiation sources, in this case two ultraviolet low pressure mercury lamp sources 250 and 251, are positioned on either side of the projector axis so as to irradiate the control surface from the rear, and to irradiate physical control details if present as well. The material of the screen and control surface 220 is chosen to be as transmissive as practicable to the short wavelength (250 nm-320 nm typically) ultraviolet radiation such that sufficient radiation reaches the front surface contaminated by a user thereof, and kills the germs on that surface within a suitable time period to suit the application. If discoloration of the plastic screen member takes place due to this irradiation, the screen and control surface can be easily interchanged as noted above. This is facilitated as typically sensing is also done electro-optically (using means disclosed in previous cases (such as camera 215) with no wires being needed between the computer and the control surface

While the rear projection device housing is deeper than competitive LCD or other flat panel devices, I believe that use of sterilizing radiation with a rear projection system as disclosed is better than trying to illuminate an LCD or other flat panel device with UV from the rear, as no degradation of the image generation materials such as liquid crystal, OLEDs or the like occurs and there are fewer members between the radiation source(s) and the front human contact surface which could absorb or otherwise denigrate the sterilizing radiation In addition, with such a rear projection device there are no active elements such as liquid crystal materials which can be interfered with as well.

The UV radiation in this rear projection case passes directly through to the front of the screen, which in the case shown has dispersive elements 222 at or just beneath the surface. This the radiation is much better able to act on germs which are on the surface. And powerful UV radiation can be used which does not hurt any function of the display device or void its warranty or that of the touch and control sensing system. By using optical sensing as well, one does not disrupt delicate capacitive or other overlays placed on the screen surface or behind it, as required with a LCD or other flat panel touch device.

It is often desired with rear projection systems to use a Fresnel lens such as 260 (dotted lines) to collimate light from the projection source in order to achieve uniform dispersion from the screen. In the case of this self-sterilizing apparatus it is however, less desirable, due to absorption by the Fresnel lens of UV sterilization radiation. If a Fresnel lens is desired for projected image improvement purposes, it is desirable to make it as thin as possible. Such a lens can serve as a front window for the system, when the screen and control surface is removed, and thus helpful to keep foreign matter out of the housing during that period.

The use of two radiation sources rather than the one shown in the aforementioned FIG. 12 of the '165 co-pending application allows a more thorough coverage of the screen, and allows more light to reach the sides such as 255 and 256 of the knob 221 or other physical controls such as pushbutton switch 265 where the person touches.

It is also noted that due to the diffusive properties of rear projection screens, that uv radiation hitting the screen from the rear will further disperse in angle, creating still more irradiation angles of the physical control and the front surface (assuming dispersive elements are in the screen material or located on or to the rear thereof).

The apparatus just disclosed is not limited to use in Operating Rooms and it is envisioned that this layout could be expanded to all instrumentation in a hospital for example, with such a controller serving to keep all equipment effectively germ free. The use of the self-sterilizing feature can be in addition to normal wipe down procedures, since another advantage of the rear projection screen and control surface is that it can be cleaned easily using many types of chemicals. If cleaned regularly, the UV radiation time or intensity or both can be reduced in some cases, saving potential degradation of the screen and making safety issues less needed to protect passersby from UV radiation. For example, one can energize the lamps only via a suitable interlock, when a cover is placed over the screen and control surface. One can also use a proximity or motion sensor to sense if anyone is near the unit, and energize the lamps or other radiation source only when no one is present.

Radiation sterilization action can be energized after a certain time period has passed, or after every actuation of the touch and/or physical controls, or as a result of some certain type of operation performed by the user, or some other criteria desired. In one example of the latter, the computer stores information concerning the user activity and when the screen has been determined to have been touched three times in one hour, the sterilizing radiation is activated.

In another example employing an interlocked protective cover which can slide over, or otherwise be placed in front of the control surface, the user is warned that the system cannot be used for its intended control purpose until the cover is so placed, and a sterilization cycle is undergone. Thus suitable cleanliness standards are enforced.

While two UV low pressure mercury vapor lamps have been shown, it can be appreciated that to illuminate from more angles, that additional lamps on either side of the projection axis in the direction in and out of the figure would also be desirable where possible and affordable, employing two additional lamps in this case. Alternatively, one may also provide a circular ring UV lamp, generally substantially centered on the axis of projection lens. And one may provide a larger number of point type sources as well. For instance UV sources other than mercury vapor lamps can be used, for example UV LEDs or Excimer lasers whose beam can be scanned in order to illuminate the control surface with non-uniformly distributed illumination if desired, dwelling for example on more contaminated areas where touch has occurred, as sensed by the camera 215 or other means. Such sources may be controlled as well to irradiate zones receiving contact by humans with more radiation than other zones which have been sensed as having not been touched as much for example. This can reduce the UV radiation requirements.

A big advantage of the total system, is that control panels can be built suited to all of the instrumentation in a hospital, and each one can be self sterilizing. This achieves a major economy of scale. Even the vital signs instrumentation taken to patient bedsides can be so operated. The layout of physical controls on the screen and control surface, and the touch screen graphics and sensing is able to accommodate a myriad of different functions just by changing the graphical interface and the screen and control surface.

In addition, And as shown herein, the unit can also uniquely sterilize the user contact surfaces of any physical controls utilized. Such controls are preferred for many medical tasks due to their intuitive feel and understanding by diverse user groups. As will now be shown, controls attached to the screen can be sterilized, such as knobs and switches and joysticks of compatible design. The knobs can be illuminated so as to redirect energy to their gripping surfaces which may point normal to the surface of the screen for example. This is an unique characteristic of the invention. In some cases it also allows one to irradiate zones with knobs more than others, as the knobs (or other physical control details) can act to shield the radiation from passersby.

FIG. 2b illustrates a close up of irradiating two physical controls, knob 221, and push switch 265, UV LED 267 irradiates switch 265 while UV Led 269 irradiates knob 221. Camera 270 senses both knob and switch positions or states as taught in copending applications. In the example shown the diffusing portion 283 of the screen and control surface is optionally either reduced or eliminated in the center of the controls, and the irradiating energy more directly strikes the surfaces touched by users thereof. In the case of the switch, it is the front face 268, while in the case of the knob, it is the side walls 255 and 256 which are impacted by virtue of the 45 degree cone 272 within the knob. Once again, irradiation is energized until a suitable time to effectively eliminate contamination from the touched surfaces.

The light weight nature of the screen and control surface, generally devoid of electronics, not only allows easy interchange, it also allows one to easily move the screen and controls easily out of its fixed position and into an area where they can be treated with radiation. For example consider FIG. 2C in which a system 285 has screen and control surface 286 having knob control 288. This is projected on by projector 290 and finger touches and control sensed by camera 291. For irradiation, the screen and control surface is flipped down into position 298 (dotted lines) for irradiation by UV lamp 293. Additional lamps or UV LEDS 294 and 294 may optionally be employed to ierradiate the user contact surfaces of knob 288.

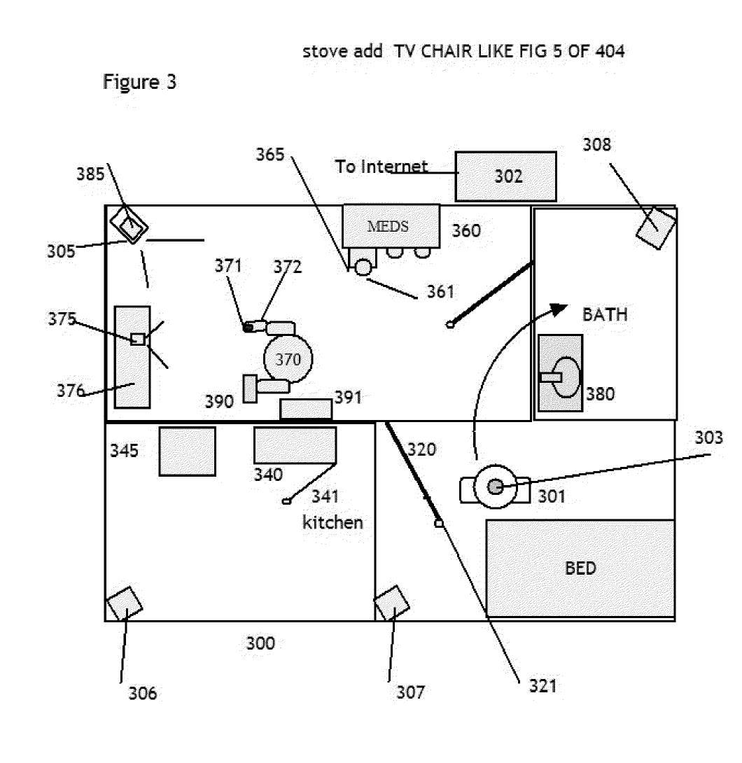

FIG. 3 illustrates a camera based control system for assisting persons living at home or in another facility. It is also useful for improving the well being and health of the mentally ill and other disabled persons as well as others who need some assistance in monitoring their daily life and often benefit from interaction with care givers and health care professionals such as family, case workers, physical therapists and others. In the examples now illustrated the camera system invention is used in much the same manner as disclosed above, but to monitor the location of simple near IR reflecting targets on the person in the room. (Alternatively in some cases colored targets having high contrast can also be reliably used) To a degree at least the system does not intrude on privacy as the camera is set up with an infrared filter so it cant take pictures of persons themselves due to the low light levels of all but the infrared reflection from the targets. This has many advantages, primarily in that it provides a very low cost solution to common living at home problems. For example consider small apartment 300 with 4 rooms. A central computer, 302, or a computer on a remote server, continually monitors the 4 (or more) cameras 305-308 in the 4 rooms, located so as to view the room, at least in the areas of use. In this example, the cameras are wirelessly connected to the computer 302, though they may inexpensively be daisy chained fire-wire cameras for example, easily interfaced to the computer.

The goal is to track one or more features (typically but not necessarily high contrast artificial targets) on the person in an accurate manner in order to see, for example; falls; deviations from normal routine, such as path to the bathroom, frequency time of day; abnormal movements such as unsteady walking, head nodding too much. The cameras can also be used for security purposes. And the invention can not only see the movement of the person, but also the movement of objects within the space, such as the opening of doors, the lifting of toilet seats, the use of kitchen utensils and so forth.

As pointed out in previous referenced applications, one can have retro-reflective material or highly contrasting colored material for example, around or attached to portions of one's body or objects associated with you. These include a collar around one's ankles and wrists a headband or one can also make the target material such as Scotch light 7615 into part of one's clothes for example, outlining certain areas, simply acting as decoration and so forth.

A simple application is where a person 301 in the house is wearing a hat or some other thing like a hairpiece with a single retro-reflective target 303. This target is visible by cameras 305, 306, 307 and 308 located in the upper corners of the rooms of the apartment. When the person is not sleeping. Typically the cameras employ as well as Near IR light source (eg 880 nm, and not shown for clarity). The target device remains on their head, and everywhere in the house, they go. This target piece can be seen if they would suddenly fall down. The target which seen be seen to drop in the field of view, and then either perhaps not be visible at all or certainly stationary for awhile or remained stationary. This sort of us signature can be used to predict. A potential fall, and essentially sound an alert that alerts the caregiver that something may have. In this case to avoid false alarms, and having to have someone immediately come there. The TV camera changes from infrared illumination mode to visible light illumination mode and a regular image of the room is produced the first image being data for the the occurrence happened. The caregiver can then look at this image and see if something bad is happening or for that matter it to assure that the person is actually in the room. The person is not in the room than other rooms can be looked at as well in this manner.

The desire for infrared illumination is to make the whole system unobtrusive to the person in the house. It also allows it to work at nighttime when one would not like any visible lights to be lit. If the camera is to be used to take visible images too, then any infrared band pass filter used on a camera has to be moved out of the way, assuming a color image is needed. If an it image would suffice, one can just up the IR energy until a satisfactory IR exposure is made, which is sufficient to identify problems. This aspect is important too for determining if Alzheimer's patients and others suffering from dementia are not in the right place.

Given the low cost of cameras today, it is probably best to have a separate color camera for taking of normal pictures.

As pointed out elsewhere herein and in my co-pending applications 3D cameras can be used if desired to gain additional information and to allow background information to be eliminated. However for many purposes the use of retro-reflective targets allows reliable data to be taken with simple low cost equipment.

Retro-reflective material can be used for ribbing or other decoration on clothes as well. For many seniors a pendant necklace is desirable having a call button device on it. This necklace itself can be made of a retro-reflective rope or band like material at which can then be seen around the persons neck as well as from the front. And back assuming that the shirt collar does not block the view

Various types of optical signatures as a result of movement can be accurately seen and recorded in this manner. For example, one can record signatures of the normal daily activity where the person say gets up from bed and goes to the bathroom at night. This has a normal signature of movement out of the bed assuming a suitable target or clearly visible natural feature is on the person while in bed, for example as part of a pajama. The movement to the bathroom can be tracked including opening of doors which themselves can be targeted, such as door 320 with target 321 also able to be seen by camera 307. Other things that can be targeted can be anything, and particularly those items depended on for daily living. For example the toilet parts, the water faucets, various utensils in the kitchen, the range, and so forth. In short, anything that might have something to do with the behavior or safety of the person and need to be monitored in the sense of potential abnormalities in that behavior causing some sort of problem. The refrigerator door 340 is another with target 341 which can be observed by camera 306 in the kitchen.

None of the targets have to be particularly obtrusive, as they don't have to distinctly reflect visible light. And even if they are to be used with a visible light camera, they can be small and/or decorative. In the above application, lye given an example of a single target on a the top of the persons head. The point, for that location is it is visible by cameras overhead in the corners of the room. There are however many other potential target locations. Indeed one would likely have more than one target on the person. This is also in case of some obscuration or, the problem with one of the targets but also to allow viewing from different vantage points and to view different activities.

The invention is very low cost in this manner, since cameras and light sources or inexpensive and central control computer 302 can be also used for other purposes in the home and to communicate over the Internet with the caregiver's PC, and indeed some of the machine vision processing used in 302 to analyze the target or other images can also be used for other tasks as well. Another potential application of virtually the same equipment is to monitor the persons activity while exercising. This is often for rehabilitation purposes as the senior citizens are most likely to be ones recovering from some orthopedic procedure, or from a stroke for example

There are certain types of activity such as walking after getting up from sitting down for example, that can indicate certain medical problems that could be seen using the invention in this manner. Since the bed in a room can be targeted, one can also see if the person is in or near the bed, and one does not have to have a complete room image. One can also see unsteady motion of the person particularly the case if target more points on the person are targeted. It is noted that small flashing LEDs, can constitute targets too, but require battery power and are more obtrusive.

Medication dispensing unit. For those who suffer from lack of physical dexterity or impairment, and those taking multiple meds or complex med schedule there's also an application using the camera sensor to observe the taking a medication which is also a problem for many seniors in the home, particularly those who suffer from mild or moderate dementia. This is also due to the number and types of medications that they need to take at different times of the day since the computer is aware of the time of day and the camera connected to the computer can see the medication box, such as medication box 360 including doors with easily seen targets such as door 361 with target 365. It is possible to determine at what time the person came over to the medicine box and which doors were opened. If the wrong door is opened and the camera is not obscured from seeing the door some sort of a sound could be made that would indicate to the person that they were picking the wrong medicine for that time of day. This is by no means a small problem, as there might be 15 different pills at different times of the day. The Invention may not be able to do everything you would like, such as actually count the number of pills at the person physically puts in the mouth. But it can at least make a start at the problem. And to a degree might be able to do this by having a door only dispense one pill at a time. So it would then have to record that if one was to take three 350 mg pills of a certain type at that time of day that the door would have to be opened and shut three times. This, the camera and computer can see and do easily. It should be noted that this system is consider a less expensive than automated medication dispensers of conventional types.

The camera in a more sophisticated machine vision version can see the person's fingers or other datum such as a target or other feature on the persons sleeve, and note that after reaching into the box, that the person put the fingers up to at least near his mouth, indicating that the medicine had been taken. Even more accurately would be to observe the person directly using an optional second camera located to view his fingers and face, if the first camera was unable to do so, as is the case of camera 305 which is not positioned to view the face of a person taking medicine from medication box 360.

While the invention's machine vision based camera system cannot easily measure vital signs of a person, it can be used to help remind the person go to a place in the apartment where ones temperature or pulse or other variable could be measured. And it can observe that they actually place their arm in a blood pressure monitor or other machine to have the pulse taken for example. Again this can be done easily if the person is cooperative with retro reflective targets or other high contrast features. Even normal features of the person for some of these applications can be processed economically by machine vision as it is. But for monitoring motions at night and infrared as well as for making the system simple and effective, the retro reflector's are helpful.

FAIL SAFE OPERATION. It should also be noted that the system in this form fails safe. That is to say that if one cannot see the target that is supposed to be on the person, then you basically can sound an alarm (e.g. over the internet to a caregiver) to check the image manually once again to make sure that things are okay. In other words, the system cannot be reporting satisfactory information if the target is not visible. A caregiver can override the system, a remotely by simply looking at the image and can perhaps check the infrared image as well to see why the target isn't there. For example it could be some kind of a fluke where the person had for example put a flower in their hat that covers the target.

Besides having a camera switch onto a visual in case of some sort of warning, the computer can also turn on a microphone as well to record what's going on. This also can be used to listen for heart or lung sounds, if they are discernible to microphone louts sounds of letter say, costing typical pneumonia or congestive issues probably be heard. The camera system in the same invention allows a nurse who might be on the scene to transmit a high definition wound image.

Analysis of the data taken may be totally done by automated means and in certain cases can provide an assessment of the function of the person. For example, one sort of an assessment can be the time taken to get up from an armchair which can be determined if the sensing system can see a datum on the person which moves upward indicative of the function. Undue slowness, hesitancy, abnormal movements of the trunk or upper limbs staggering stumbling—all of these things can be built into an assessment program for future determination of of what is possible for the patient, as well as to simple monitor that nothing undue happens. One can also use the invention to monitor dexterity and reaction time in chores or another activity done repetitively each day.

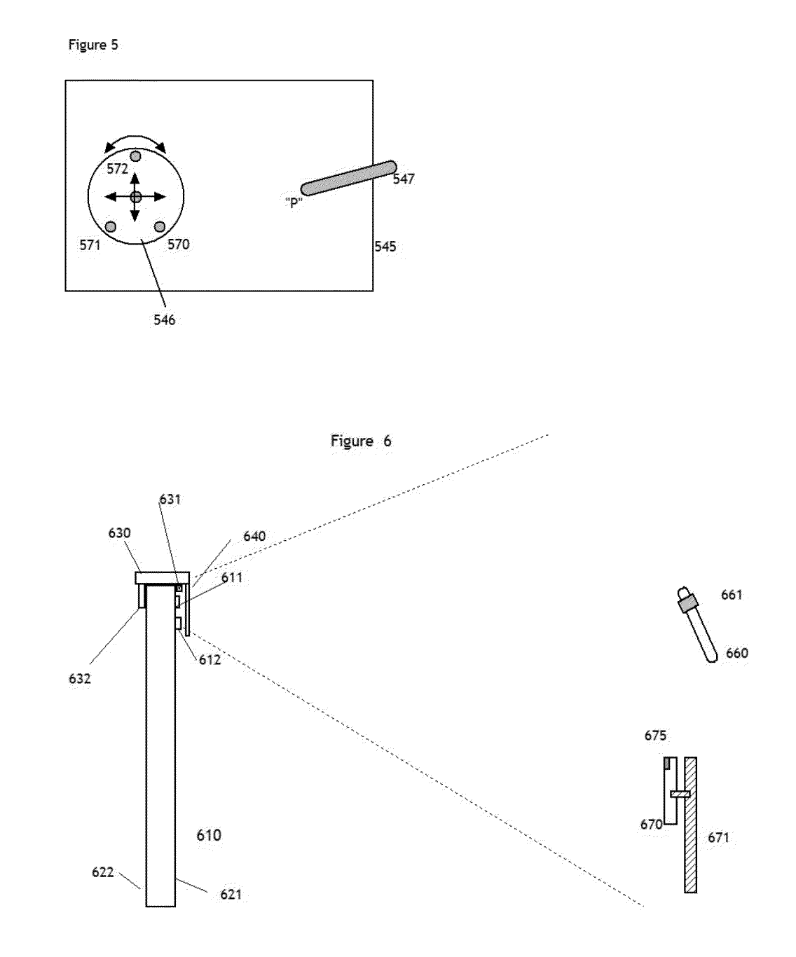

Camera systems, be they 2D or 3D, can be used for tracking the person, as well as for determining the persons relationship to other objects in the home, and indeed to the status of those objects. This can be done to assure caregivers that things are alright, or it can be done as noted above to assist the person themselves. One example is tracking the movements of a person such as 370 in exercising against a resistive force such as an elastic band or weight or a resistive machine in front of TV display 376. This may be done for example as shown in FIG. 5 or other figures of my co-pending application Ser. No. 13/668,351. Data obtained, which can also be from tracking a portion of the person and/or the object moved, can be obtained by the camera 305 in the corner of the room or from a second camera 376 for example (also wirelessly connected to computer 302 in this example) on or incorporated into the TV 307 or a set top box on it, which can determine motions and feedback help to the person on the TV display as well as allow information to be communicated with a therapist as needed. As noted above, the person's picture need not be taken in order to gather the data which maintains privacy often desired with such activity. Under certain circumstances the picture can be taken, perhaps using a separate camera for the purpose and under supervision of care givers and health care providers.

The use of the system can allow caregivers to interact even on a daily basis as may be desired with certain mentally ill patients as disclosed in co-pending applications such as Ser. No. 13/544,180.

A somewhat different application can also be performed using the system. The user is familiar with his or her TV 376 and uses a remote control with it. It is possible with the invention to attach a retro-reflector 371 to the front of the remote control 372 allowing it to become a means to move a cursor on the screen which can be used to select functions in a simple manner—augmenting the function of the remote itself, and allowing the person to interact with the computer 302 of the system as well. In this case a LED light source such as 385 is also used near the axis of the camera used. The system provides a great deal of utility for persons wishing to operate television sets while having added functions including motion sensing for therapy or games or other activities, medical monitoring and the like

Alternative to tracking the remote control or one can also track using camera 376 for example, the IR LED in the remote, (assuming the person has it turned on) to provide a means to control a cursor. Alternative to a retro-reflector target, one can add a LED (and battery) to the person or to a remote, or to an exercise object and track the LED (or LEDs) as the case may be.

The system as a whole, with infrared light sources and cameras in every room, can not only determine if atypical conditions such as unsteady walking or falls exist in order to alert care givers, but can also act to assist the person with certain problems. The taking of medication has been mentioned above, a real problem area for senior citizens and others. Lapses of memory of having turned things on is also a common problem For example and as pointed out elsewhere herein, the camera and computer of the invention can be used to sense and signal an alarm if dangerous conditions exist in the kitchen. Sensed items can be water boiling, pots boiling over, burners left on, pans in dangerous positions, fridge door left open, and the like. A camera such as 308 in the bathroom can be programmed to tell if the sink 380 or bath is filled up or overflowing and thus to signal the person if this is the case.

Another area also mentioned in subsequent embodiments is to aid the person to control items in the home. For example to control functions in the kitchen using gestures made with ones hands as well as ones voice where practical. These can be useful if mobility is difficult and in other situations. The cameras in the rooms can determine simple gestures as shown in FIGS. 17 to 19 and in co-pending applications, a process made easier if retro-reflector targets or full 3D cameras are used, either of which can reduce the effect of background issues confusing the results.

Some other gesture controls and controls using simple knobs and switches which can be seen by the simple cameras in the whole house system are now described in the following embodiments. While in the context of a hospital room, or employing mobile devices they apply as well to one's home.

To make things simple for camera sensng, auxiliary easy to detect objects may be used. Consider person 370 in front of the TV set. To tell the TV what channel to choose, the person could just pick up a special card such as 380 shown, say 5×7 inches in extent and of a given color or shape or with a distinct graphic pattern for example. This card would be for that channel. By holding up another card of different characteristic for camera viewing, one may select another channel, or another mode of operation, for example a video conference using Skype with a son or granddaughter say. Use of such a technique can make a robust signaling and control system without requiring substantial computer processing. In turn, more cameras for a given cost can be placed in the home, rather than having expensive time of flight 3D cameras for example. The uses can extend into all rooms of the house as well and serve as an alternate or adjunct to voice or gesture commands.

Note too that these simple system techniques can also work with smart phone and tablet cameras as well, keeping cost low. For example, the camera 375 could be that of a person's phone.

I feel for example, that the kitchen work surface aspect of the invention disclosed below and in co-pending applications will help as well by making meal preparation less difficult, especially for older persons who have not historically cooked. It also adds a degree of safety as activities in the kitchen area can be camera monitored and warnings given of dangerous conditions such as water boiling too long, heat left on and the like. Indeed, the system can include automatic shut down of burners and ovens if needed.

FIG. 4 illustrates another embodiment of the invention. Here a single camera 411 located in the ceiling 410 observes an area beneath. A LED light source module 412 is used in this example, which may include plural LEDs, in this case infra-red LEDs at 880 nm for example. This LED source (or alternatively a diode laser source) is often accompanied by a band pass filter centered on the same wavelength placed in front of camera 411 to maximize returning signal from objects or targets in relation to ambient light illumination.

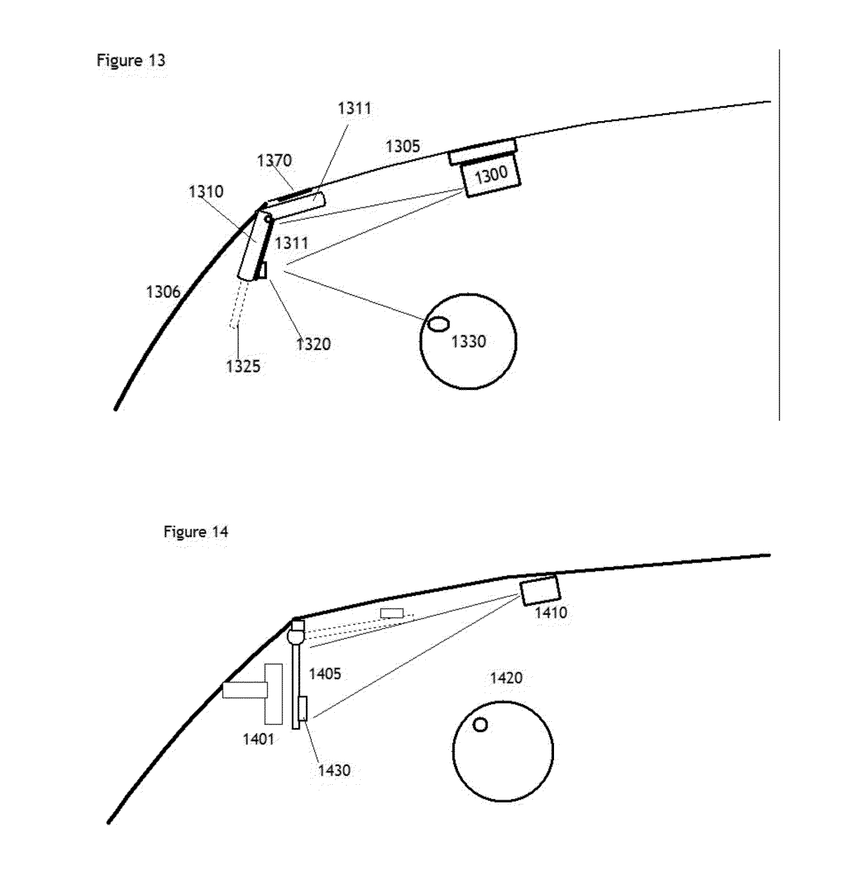

The system can be for example a camera in a room of a home similar to that just disclosed in FIG. 3 above and in FIG. 13 of my co-pending application Ser. No. 12/748,657 relative to aiding seniors living at home and other similar applications, for example in nursing homes

Let's consider a room in a hospital or nursing home. The camera 411 with LED illumination system 412 views in this example a low cost multi-axis plastic control knob such as 446 mounted to a plastic plate. The camera also can view a users finger 447 touching or over the plate. These knobs and plate are in a hospital setting and are at least pre-cleaned (and even sterilized) and placed on the arm of a chair or in this case a tray 440 attached to an arm 431 (or 432) of patient bed 430. A big advantage is there are no wires to the knob or to the vicinity of the patient's bed to get in the way or cause electrical difficulties

The multi-axis knob can be manipulated both circumferentially and in x-y as will be discussed further and the positions chosen in turn be used to control TV 455 by virtue of computer 420 processing image signals obtained from the knob from camera 411. Alternatively use of gestures to control a TV 455 in the patient room can be done as well with the FIG. 4 apparatus by sensing the position of the user's finger on or above the plane of the plate.

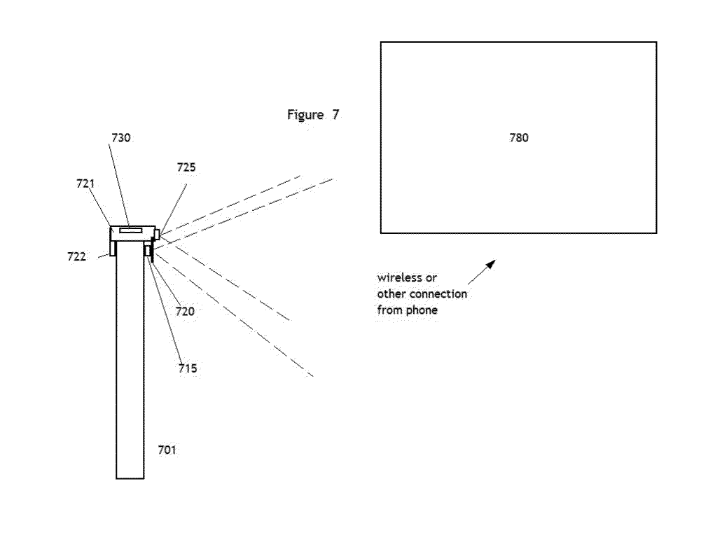

The camera can alternatively be that of the persons own cell phone as disclosed in FIGS. 6 and 7 below with the control data transmitted wirelessly to the TV. In either case one can integrate with a hospital wireless and cellular connection for safety.

A nice feature is that simple two axis finger gestures can be made by the patient or other person on the plate 445 and seen by the camera 411. This plate can be a pre-cleaned or sterilized work surface in this regard of very low cost on which the patient can make finger touch gestures or other inputs on. The plate can be changed daily, or with each patient or at some other interval. The gestures are shown in FIG. 5 on a plate housing a multi-axis knob, but the plate can be bare and just serve as a background for the finger when viewed by the camera of FIG. 4 or of the phone or other devices disclosed. The plate can also be blocked off with printed data showing common items of interest, which the person can select by touching with his finger. An obvious one TV wise would be to touch the plate to select the channel. Another would be a menu plate given at meal time having all the menu items of the day to point to. Data entered by touching the plate (or a print overlay on the plate) can be displayed and confirmed on the TV. This system is similar in a way to that of FIG. 1, but does not utilize projection of data onto the persons unit, only the sensing of input data from it, either by touch or by the actuation of physical controls such as turning a knob for TV volume.

In one embodiment a small version of the FIG. 4 device, with or without the screen, can be built into a hospital bed or attached thereto and each patient provided with a new sterile control panel for example, to operate TV using joystick for menu and knobs, or just using knobs, or otherwise. For example this can be used to call a nurse or for requesting other services or to answer a phone. Or one's own cell phone can be attached and used in a controlled manner suitable for the hospital environment. Alternatively or in addition one can use ones gestures to control a TV or other device, either gestures in space or, as noted in FIGS. 4 and 5, as finger gestures made on or over a pad or plate located on furniture, bed trays or the like It should be noted that the camera 411 can observe multiple control locations at once. For example, one could observe the inputs of two patients in beds side by side. And one can view multiple controls operable by a single person, but indifferent places. For example another control could be attached to the top of bed side member 432. A particularly unusual application is to attach the control device with or without a underlying plate to the person themselves. For example in the FIG. 4 to a person's chest being operated on, assuming for example the 430 was an operating table. This latter feature allows the control device, again being of simple sterilizable material of minimum weight and complication, to be put anywhere desired, as long as a camera can see it. For this later purpose one could have multiple cameras in a room such that at least one had an obstructed view and could be seen accurately

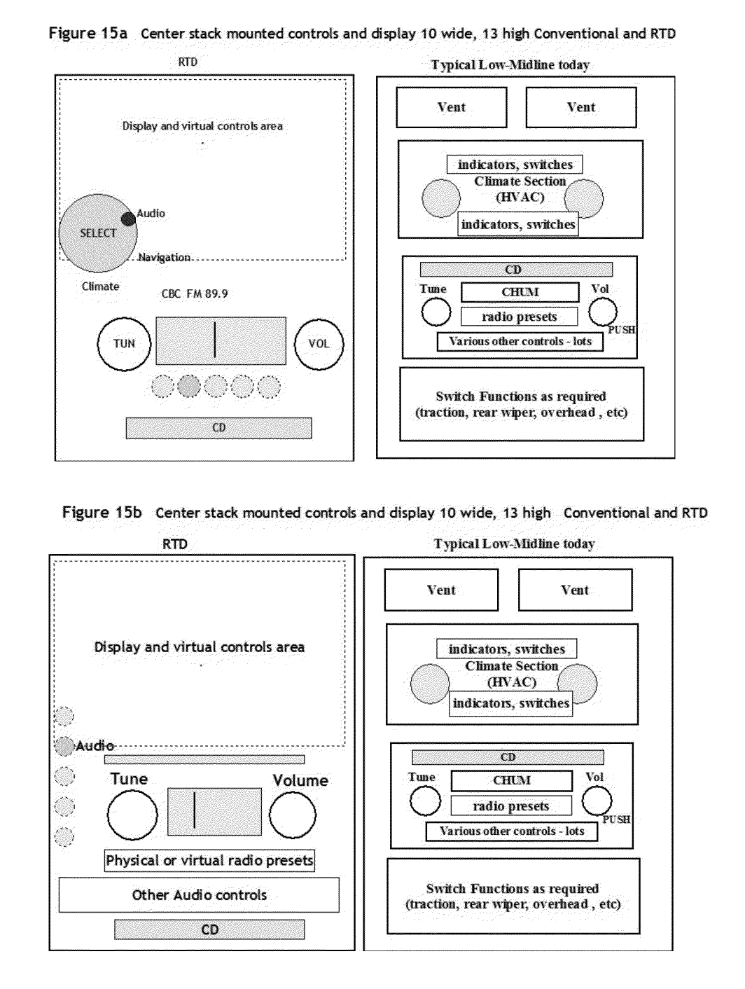

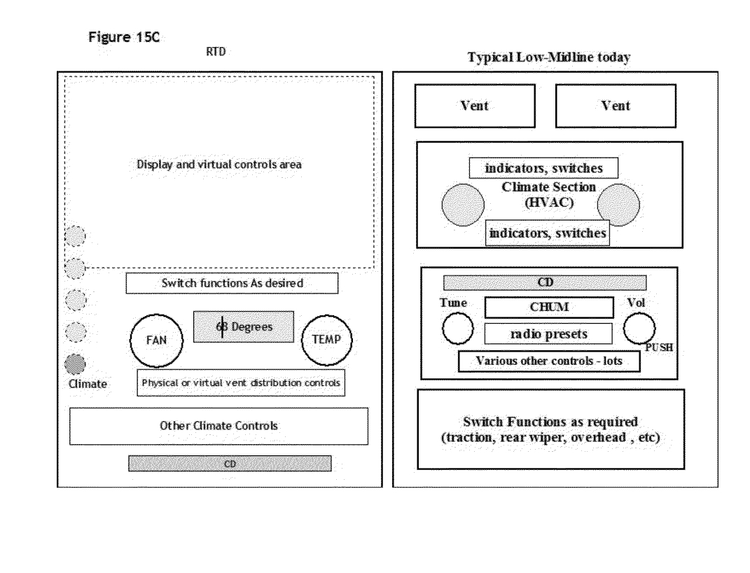

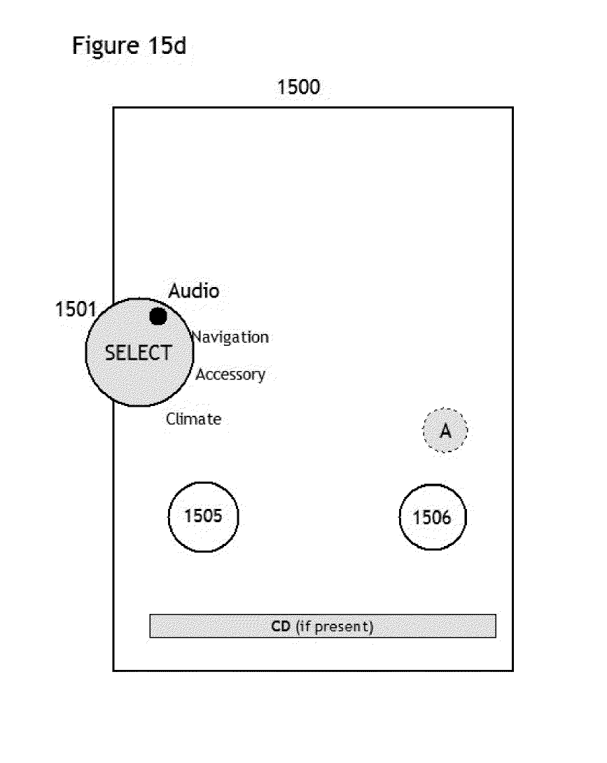

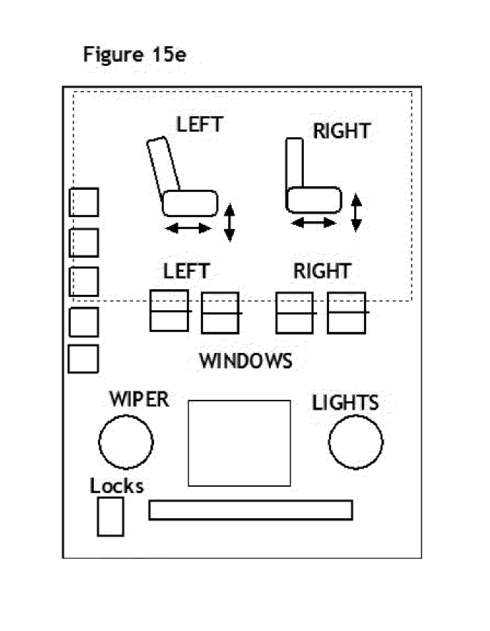

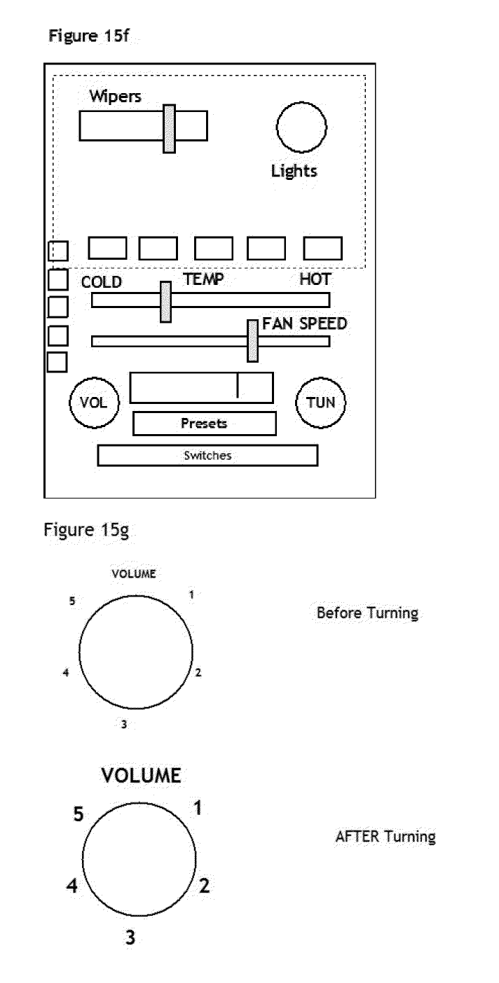

FIG. 5 illustrates further the plate 445 in a top view, along with a novel joystick type knob such as shown in FIGS. 3 and 15 of my co-pending application Ser. No. 12/897,077. In the instant case however, the knob is observed from above rather than behind. The plane of the paper is the x-y plane, the direction of the camera is z.

This simple device can control TV or other device all by itself using the x-y direction of the knob urging to select from a screen menu, and the rotation of the knob to change volume for example. It can be used in hospital, or alternatively in the home example of FIG. 2. In this example of operation, the rotational position of the knob is indicated (in this one example of how sensing by camera 411 can be achieved) by the axis of the isosceles triangle formed by three reflecting targets such as 570-572, and the x-y location in the plane of the knob is determined by the center movement of the triangle so formed.

It is also possible to sense other features of the knob to obtain rotation and x-y location. And it is possible to do this from overhead, rather than from behind. For example one may track the rotation with a single dot on the knob circumference facing up, and a single dot in the center of the knob can be used to sense x-y location using a camera located overhead. The person when gripping the knob simply has to take care not to obscure the datum's employed. Because of this problem a ring around the whole knob (which could be the knob periphery itself) may be used from which one can solve for a center point even if parts of the ring are obscured for example. Alternatively the center of the knob proper can be sensed to determine x-y movement. A high contrast image of the knob and its periphery is obtained if the knob face is retro-reflective say, against a non retro back ground, or visa versa.

If the axis of rotation of the knob is secured to a bendable portion of plate 445, the knob can be a multi-axis device and be moved in the x and y direction for a small amount in each direction, as well as rotate about an axle or other bearing attached to the plate. It is not necessarily required that the axis spring back to a position at right angles to the plate, since movements on a TV screen for example are often relative to what a cursor position on the screen is at the moment.

The plate 445 of FIG. 5 can itself have on its top face retro-reflective flat material such as Scotchlight by 3M. or Reflexite. The plate can have a smooth coated or plastic surface for protection and ease of finger movement if desired. This may be achieved as well with a plastic window surface over the retro-reflector. The use of the retro-reflector of the plate allows the maximum returned signal to the camera and results in a high contrast dark image of a finger moved on the plate, which finger image appears dark as nothing is retro-reflected from it. This allows one to easily sense the finger tip point P with the camera and signal a gesture or other indication to the computer with your finger. Such a device can be used if desired for other reflective touch gesture applications as well. One also can use objects of any type to provide high contrast dark signals which indicate something either by their shape, juxtaposition or other criteria. It is also possible to use disposable sterile knobs and other physical controls by themselves, not necessarily provided in connection with an underlying plate member 445.

If one is laying in hospital bed, or your bed at home and don't want to use a conventional and notoriously dirty remote to control a TV or other equipment, the invention may serve as an excellent alternative. The simple knobs, plates and joystick knobs can be attached easily to anything and removed easily, using Velcro, light adhesive or the like. A knob attached to the arm of a chair for example can be seen from overhead easily if the targets used to determine its rotational or xy position are at top of knob.

It is noted that the device can function with simple webcam cameras usable in phones, or the like. (noting that IR LEDs can be best employed if any IR blocking filter present in some webcams for photography purposes is removed) It can also function with more sophisticated 3D camera systems capable of determining the range to the target as well as its location in the x-y image field

It should be noted that because the entire plate 545 including the knob is low in cost, it can be disposed of after use by a patient. Alternatively it is also easy to clean or sterilize as there are no electronics, only plastic typically (though other materials may be used).

FIG. 6 illustrates a method and apparatus to sense objects, such as persons, controls or the like which is an advantageous modification to smartphones and other devices which employ white light flash or video illuminators, such as 611 shown in smart phone 610, for example an iPhone by Apple Corp. This smart phone has a camera 612 and, typical of such devices, the camera with a near infrared blocking filter incorporated. Thus near infrared illumination of reflecting targets such as retro reflectors described in my co-pending cases cannot generally be effectively used with this camera. The use of near IR illumination is a desirable feature in that bright sources can be used without blinding or at least overly distracting a user positioned in front of the device. The invention of this disclosure overcomes this problem for many applications and provides unobtrusive illumination of persons and other objects by using a wavelength based (and typically, but not necessarily, one color) filter in the visible region, the filter having for example a pass band of for example, 30 nm to half power points. This cuts the outgoing white light (which generally is of high intensity for photographic purposes) from the LED by about 10 times (in one example), thus making it less obtrusive. The returning light from the reflector however passes right back through the filter with minimal attenuation as the filter is has substantially the same characteristics for both outgoing and incoming radiation. While the light used for sensing is cut by the same 10 times (over the amount of white light available), the ambient light in the room is also cut by 10 (all numbers very approximate). Thus the signal to ambient noise remains approximately the same, but the obtrusiveness to the user is much less.

Images from the camera are typically processed using machine vision software such as opencv for android or ios to enable reflector positions or movements to be determined. In the simplest case one can threshold the image and look for the centroid of bright signals above a threshold, indicative of returning light from a retroreflector in a moderately lit room.

As shown in FIG. 6 indicative of one example of device construction, there is provided a U-shaped bracket 630 having two portions 631 and 632 fitting over the top of the phone (and clamped if needed for example by a set screw, or the elasticity in the bracket), such that a filter 640 can be positioned in front of both the white light LED used 611 and the camera 612. The band pass of the filter used can be chosen to vary the amount of light admitted. If this filter is for example a relatively monochromatic one, the scene illuminated will be generally dark in normal room light condition, while the returning light from a scotchlight or other retro reflector such as 661 will appear bright. Alternatively, if more image of person such as the persons finger 660 having the ring retro reflector 661 on it is desired, the filter can be made chosen to be less monochromatic, and accordingly light distraction increased.

It is noted that one can improve signal to noise however by also using the color sensing and processing of the camera 612 and the computer of the handheld or other device. In this case the particular wavelength returning through the filter is also looked for in the camera image, to allow a further degree of signal refinement and rejection of ambient light not in the wavelength chosen.

Indeed there may be some cases where the additional filter in front of the camera can be dispensed with entirely, and the camera processing itself used. For example, if a Red filter is placed in front of the white light LED 611, and the filter 640 does not extend far enough to cover the camera, one can use the cameras own Bayer or other filter system to determine the presence of the red wavelength in the image of returning light from the retro-reflector, while filtering out light at other wavelengths. Often though it is desirable to use both filtering mechanisms to improve detection in strong ambient light situations.

It should be noted that the advantages of reduced light to disturb users is also achieved if the target is an active target (such as a monochromatic LED on a ring in place of the retro-reflector ring 661 of FIG. 6. In this case no LED illumination from the phone is required. The camera can either look just for the LED color, or as mentioned above, an added filter passing this color can be placed over the camera Such an active LED target is advantageous therefore, but at a cost of requiring a battery or other power source and is also less able to be sterilized and disposed of easily. The LED angular divergence needs to be optimized for the application as well, requiring a lens which can stick up and cause possible mechanical problems. And unlike the ring 661, the LED cannot in general be viewed from the different directions that one would point the finger (multiple LEDs can be used pointing in different directions in this case, but at a cost of further complexity).

It is also possible to combine a LED target with a retro-reflector target. For example in the ring 661 just consider adding one or more LEDs to it operating at the same wavelength as the pass band of the filter (or in the FIG. 7 case the LED illumination wavelength).

Also shown in FIG. 6 is another retro-reflector 675 on a rotary knob 670 attached to member 671. The rotational position of the knob can be sensed as taught in co-pending applications and further described also in reference to FIGS. 4 and 5 in the context of easy to clean or disposable sterile physical controls such as knobs, joysticks, switches and sliders.

In the same vein let us consider FIG. 7 which illustrates a smart phone, tablet, laptop or other device which has no LED source of its own. The invention employs an attachable LED (since many Tablets such as iPads have none today) and integral filter in front of the camera. Such an auxiliary Led is useful for the invention as LEDs are today generally not provided for front facing cameras viewing a phone user for example, a desirable location as the user can view the phone display and operate the computer within the phone with gestures using the invention.

In this case the phone, tablet (or computer or other device) has affixed to it a bracket 710 having a monochromatic LED 720 driven by battery 730 in this example collocated with the bracket. A band pass filter at the monochromatic wavelength 720 is attached to the bracket such that it covers the camera 705 of the device. Alternatively this filter can be stuck on to the phone or tablet with adhesive (or other means), if as in many cases, the surface of the surrounding face of the device is flat or otherwise amenable. It should be noted that this arrangement can work to sense the users own fingers or other object of a user looking at the display 760 of the device. While the LED is here shown operated by a battery 730 built into the clip on housing, power for it can alternatively be provided by a USB or other connection to the tablet, where it runs from the tablet battery or other power source.

It is noted that this added LED arrangement can also be used in devices such as the smart phone of FIG. 6, where it is desired to use a single color auxiliary LED to save battery power or for other purposes. In this case the filter passes returning light of the LED color. As in other examples, one does not have to employ a retro-reflector and such LED and filtering steps can be useful for general illumination where desired to limit returning light to that largely at least created by the LED(s)

The use of the filter in FIG. 6 or 7 has another good effect in that for some phones and other devices it forces the LED control of the device to use maximum LED intensity, and camera control optimized for that, for example at a longer integration time.

Some “White light” LEDs are composed of three individual LEDs Red Green and Blue which are mixed together to get white. In this case one of the LEDs can be individually controlled so as to be the only one energized when retro-reflector viewing is desired. In this case the pass band of the filter over the camera is chosen to match the energized LED wavelength. Or just the camera processing alone (no filter) is used to look for return signals of this wavelength. In this case one can use white light for general imaging, but when you want the retro-reflector signal, you just energize the red LED for example and the computer processing of the camera looks for that red color. If added dynamic range is needed, a red filter of the same wavelength can be placed over the camera

While more costly in general, It is noted that a plurality of illumination wavelengths can be used, as long as the filter used passes substantially only those wavelengths and rejects most of the remaining spectrum

An application of the devices of FIGS. 6 and 7 is to the gesture based control of the smartphone or tablet used. One popular application is games, where one can play using ones hands or fingers using the invention, for example having a retro-reflector on ones wrist or finger(s), noting that more than one object can be sensed at a time. Other objects with retro-reflectors, or with self-luminous targets at the desired wavelength as noted above can also be used. Many applications of these sorts have been disclosed in my pending and granted patents, all of which are incorporated here by reference. The invention will function without specialized targets such as retro-reflectors. However A big advantage of the disclosed invention when such targets are used, and especially for handheld devices, is that the power requirements for the computational effort to reliably sense retro-reflective targets is much less than would be required using normal machine vision using the features of the object itself, without the assistance of the target. This is especially true where 3D sensing is used—an area where simple targets can be a big help, either to a single camera, or a stereo camera pair (which has appeared in some cell phones already) or a time of flight sensor such as that of Canesta corp (now owned by Microsoft). Use of a target, simplifies the 3D sensing job particularly in unknown and not previously modeled situations.

In many cases it is anticipated that phones such as 760 will communicate with a larger display such as a HDTV 780 via cable or wireless means (e.g. Apple Airplay) for many gesture based and other activities for example those played by person gesturing in FIG. 46.

It should be noted that invention has described using a monochromatic filter over both camera and white light LED (or LEDs or other source). It is possible to have a filter having more than one wavelength however. For example two wavelengths, deep Red and deep Blue. In this case the filter in front of the camera accepts the same wavelengths as transmitted and thus reflected by the retro-reflector. This allows one to color code the reflectors, accentuating one wavelength or the other to indicate which reflector is returning data or for other purposes such as identification of target presence or type.

It is generally desirable to use a LED or filter wavelength different from other light sources in a room such as LED telltale lights on equipment. Typically these are Red. It is noted the LED and camera can in the phone or other handheld device of FIG. 6 or 7 can be sideways disposed or any other way

Machine vision software for the above embodiments and other embodiments herein can advantageously be provided without license fee using OpenCV (opencv.org). This software will run on windows, IOS and Android systems at least. In addition other software for cell phones running on the Qualcomm snapdragon processor is available.

I would now like to discuss features of my RTD invention of use in vehicles, homes and hospitals and other locations.

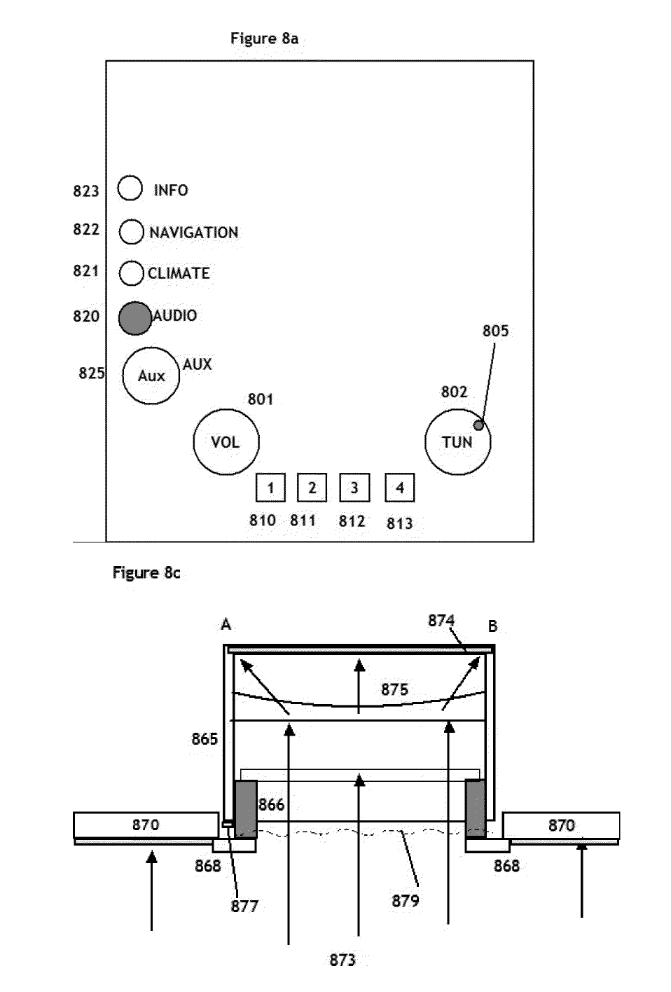

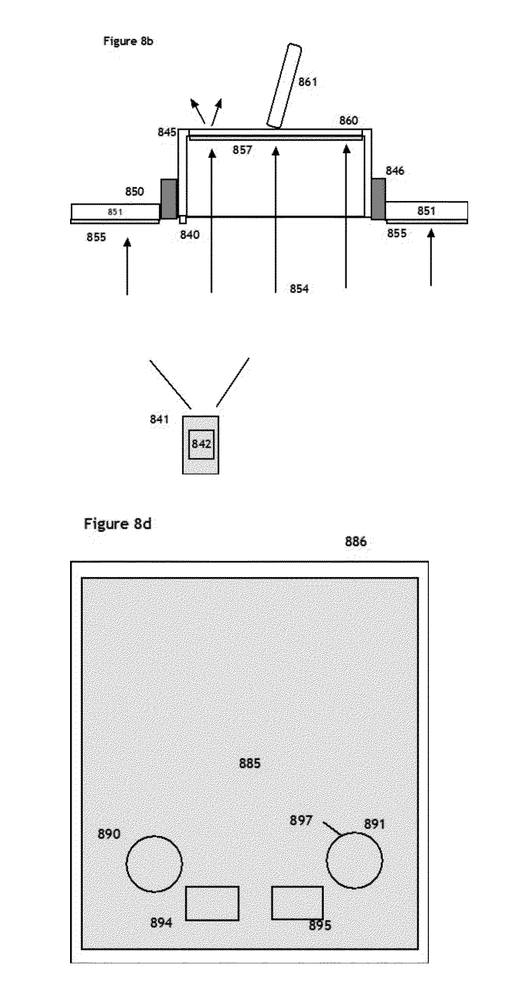

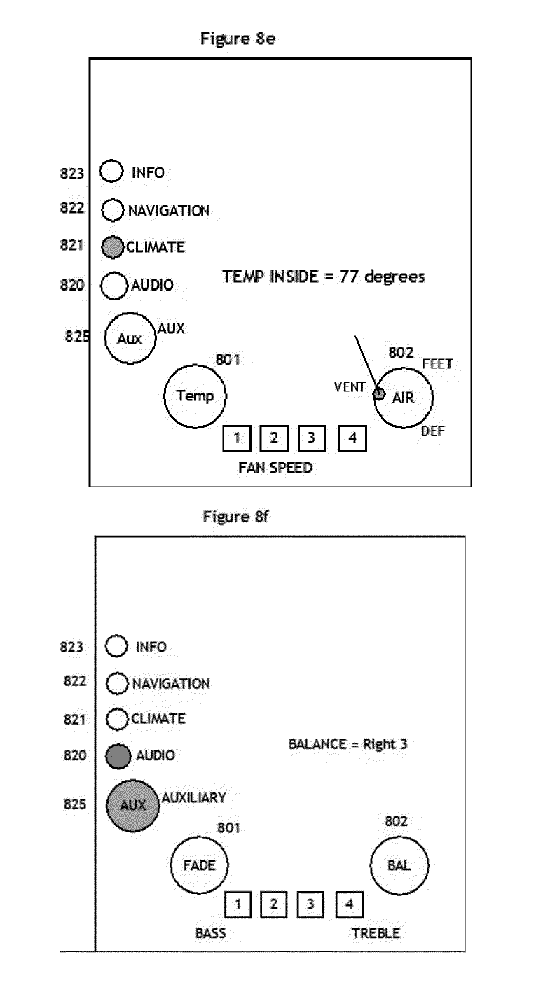

FIG. 8a illustrates a typical control surface an RTD type shown in co-pending applications and elsewhere herein, in this case having two knobs 801 and 802 and 4 touch switches 810-813 for various functions, for example radio presets. The knobs might at one time be programmed to display VOL (for volume) and TUN (for tuning) of a radio, when the corresponding AUDIO switch 820 was pressed. At another time, they might represent temperature or fan speed when the CLIMATE selector switch 811 was pressed. The function of the controls can be changed by pressing one of the 4 (in this example) selector switches 820-823 together with the function of the auxiliary switch 825. For example, the knobs might at one time be programmed to display VOL (for volume) and TUNE (for tuning) of a radio, when the corresponding AUDIO switch 820 was activated. At another time, they might represent temperature or fan speed when the selector switch 821 for CLIMATE was pressed. While the switches can be virtual icon based as described in co-pending applications, in this example, they are physical switches that are depressed into the plane of the surface or slid along the plane of the surface at the switch location. The function of a switch or other control (such as a knob) can be projected next to it, such as the words Navigation, Audio, Climate etc shown. Alternatively or in addition, each of the switches and knobs may be provided with a clear center and a diffusing surface presenting the image, such as shown in the AUX switch 825. In addition, one can project images on the face of the knob or switch which can correspond better to what is intended than say some universal pictograph. Or you can project in any language desired. This is very useful where vehicles are sold in different places, or where one has a vehicle with different drivers. And the picture projected may be personalized on the control. For example, if a knob is used to select songs from a play list, the album cover for that song, or the singer's picture, may be displayed on the knob face to facilitate driver recognition of the song without reading written words.

FIG. 8b illustrates design of an exemplary knob such as 801. As shown a target 840, such as a retro reflector is observed by a camera (not to scale) 841 equipped with substantially on axis LED illuminator 842 and its position determined, in order to determine the rotational position of the knob. More than one target on the rotating portion of the knob 845 (which rotates within the outer journal 846 which is inset into a hole 850 in screen 851 and fixed there to) can be used for this purpose if desired, which typically improves accuracy of rotation detection. Alternatively, the knob may rotate about an inner journal bearing for example. Retaining means to affix the knob to the outer journal is not shown for clarity

The screen is illuminated by a projected image information 854 which diffuses in this example from beaded diffusing screen material 855 which has been removed beneath the knob to allow projected light to pass thru the center of the knob and hit and diffuse from material 857 on clear disc knob face member 860.

It is desirable where possible that the target be seen by the camera in such a manner that light does not have to pass through the screen diffusive material 855, This allows a strong target signal to be seen, and the use of a retro-reflective target 840. It should be noted that the journal 846 which is sunk into the screen, desirably blocks light from the projector which is coming through in the region where the Vikuiti or other diffusing material is not provided on the rear of the screen to allow passage to the knob face.

Projection of knob label or other information on the knob face 860 provides a very desirable advantage in that the labeling of the knob (or other control such as a switch) can be achieved by projecting the label right on its face, thus not using up the space around the device for this purpose.

It should be noted that icons, graphics or other information presented on screen knob face 860 can also be touched, for example by users finger 161, and this touch sensed by camera (or other electro-optical sensor) 841 as previously disclosed in co-pending applications. Thus a knob (or switch or other control of this sort can also serve as a miniature touch screen. If desired this function could be provided by more conventional means, also including sensing force on the knob, or on the control surface containing the knob, as previously disclosed.

FIG. 8c illustrates a knob 865 rotating around an opaque inner journal 166, which is fastened to a ring member 868, itself fastened by adhesive or other means to screen and control surface 870. Retaining means to affix the knob to the inner journal is not shown for clarity. Diffusing material such as Vikuiti to diffuse projected light 873 is provided on face member 874 and screen member 870 as in FIG. 8b. Ring member 868 is made of a Rohm and Haas brand plastic which is substantially opaque in the visible, but transmits well at the 880 nm wavelength used in order to allow one or more reflective targets 877 on the back of the knob to be seen by the camera (or other electro-optical sensor) in such illumination. Provision of the lower ring member stops the visible projection light from leaking through the screen bothering the user.

Also illustrated in FIG. 8c is the optional use of a diverging fresnel (or other) lens 875 inside the knob in order to spread the projected image 873 to the edges A and B of the knob face member 874, which has beaded film or other diffusing material thereon. This allows the maximum projection area on face member 874. And it allows one to project whole images on the screen even in the presence of knobs, since projected sections of an image can be modified in the computer controlling image projection, so as to be correct size when expanded by the diverging lens 875, substantially and effectively avoiding the obstruction of the image projected due to the components such as bearings and the like of the knob. The same holds true for switches such as shown in figures below, which also may be similarly equipped to diverge light to their face members.

The arrangement of FIG. 8 maximizes the size of images which can be displayed on the screen/control surface including the knobs and switches. It allows the projections of complete large scale images on a large portion of the screen/control surface wherein only segments go through each knob or switch, but the conglomerated whole looks like an over all large image.

FIG. 8d illustrates the ability of the invention to display very large images in the vehicle center stack or elsewhere. An image obtained by a rear view camera or a stored map image 885 is displayed on screen and control surface 886 which includes knobs 890 and 891 and switches 894 and 895. Each knob and switch itself can display its portion of the image. While the edge regions such as 897 for example relative to knob 891 where the control meets the surface are typically obscured, the over all effect of the huge (by car standards) and largely uninterrupted display is preserved and is very useful and dramatic in its presentation. The labels such as VOL or TUN provided for knobs in normal operation would for example be eliminated in a back up mode when just rear views are desired.

There are many possible arrangements of controls of the FIG. 8 example. For example, The whole control surface can be dedicated to climate control functions as shown in FIG. 8e, by pushing climate button 821

In another example, shown in FIG. 8f, pressing the AUX button 825, in addition to one of the function selector switches (E.g. AUDIO Switch 120), may cause a related sub function to be activated. Typically this would be just to set something, like Speaker Fade or Balance (which value can be displayed on the screen, for example as shown), or a Tone Control such as bass value. These could be individually displayed as Speaker and Tone screens (for example by pushing the AUX button a second time the Tone screen would come up) or both together as shown. Then in a preferred mode, after a time delay the screen would default back to the main function selected (e.g. audio system). A control surface with two extra knobs above the radio can alternatively be provided for example too.

The value to which a knob points can be programmably varied when one switches screens, even though the knob physically is in the same place—an important advantage. For example the right knob 802 has its pointer 805 displayed at the 2 O'clock position in the AUDIO screen of FIG. 8a, but in the 9 O'clock position in the Climate screen of FIG. 8e. This is because as the user changes screens from AUDIO to CLIMATE, the current climate position for that control is displayed, which in this case is in the Vent position due to a previous move by the user, or some automatic function which might be operating.

I would like now to disclose further details on the improved switch design described above, which is based on the slider in FIG. 10b of my Ser. No. 13/613,679 co-pending application. In this case the switching action, like that of knob 801 and 802, is in the plane of the screen and control surface, rather than be pushed into the screen like a push button switch. The change in position or light change caused by the switch movement by the user is detected by a TV camera as before, and analyzed by a computer which generates suitable control commands to the device controlled, and suitable graphics display commands to cause appropriate labels or data or other information to be displayed to the user. Because it is all computer controlled, a switch of this type can serve different functions at different times, and hence is reconfigurable, or multifunctional.

This switch design makes it easier to seal the screen and protect the optics behind, as all movement is in the plane of the screen and can be seen looking through the screen, without necessarily cutting a hole in the screen, though one can do this if desired as shown in the referenced patent applications.

It should be noted that a big diameter knob allows two beneficial things. It allows more data to be displayed in its center in a visible manner, and it allows a bigger device to grasp. The invention makes all of this possible by having the labeling on the knob rather than around it, and in addition, by reconfiguration allows more space to make the knobs of bigger diameter in a confined center stack space. One can have knobs or sliders with detents or continuous motion.

As also pointed out in my co pending '679 application, a dual knob with an outer and inner ring can be used. This allows more ability to control various variables, without adding switches or further knobs. For example when you turn the outer ring (lets say that is a secondary function) the primary label in the center of the knob can change to the secondary function, and then after the control move is accomplished default back to the primary label. Labels can be written text, but they can be pictures or even dynamic video images. These images could be chosen to be examples of what makes you like operate the device for example. Really personalized, in other words. Note that one can have an appropriate label on the knob face or labeling next to device or both, if desired. As noted, these labels can if desired be in different languages, or in more definition, or more detail or some other aspect.

Switches and knobs such as those disclosed above may not only have reconfigurable text or graphics displayed but also reconfigurable display simple colors or patterns in a decorative sense, or to provide increased user understanding of the controls. I would now like to discuss the use of colors or patterns to code, in the simplest case, one of switches could be a matching color to a related knob function. This color can be projected and can be thus changed programmably to relate at a further time to another control device as desired. This color-coding is programmable and can be done for various reasons and at various times, in various ways to aid operation of the controls. This is particularly valuable with inexperienced users, such as senior citizens or others who might be confused by a multiplicity of functions in a control panel. Even for technically astute operators, the color codes allow faster reaction times.

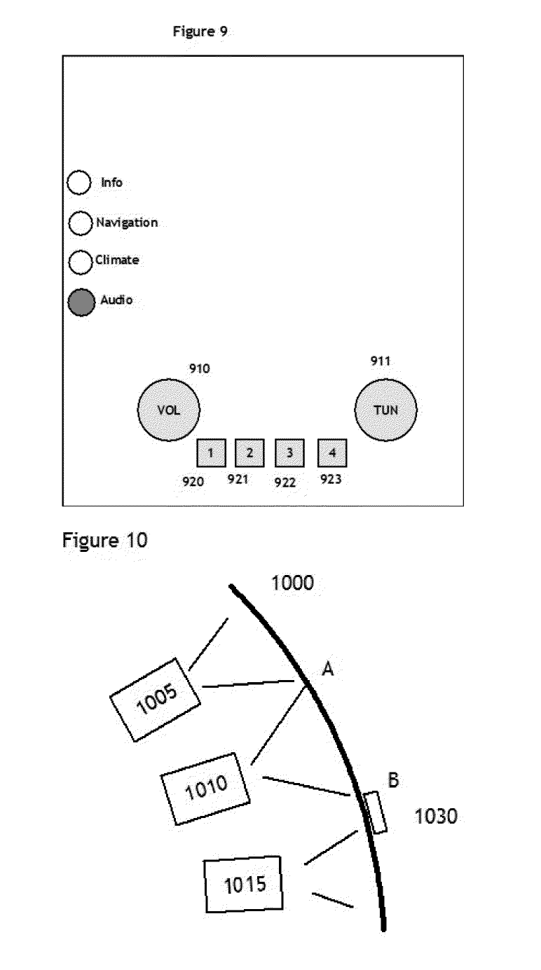

FIG. 9 illustrates a layout of the center stack 900 of an instrument panel of the invention in which six of the controls on the control surface have been color coded by a program in the computer controlling the projector (or other display), to enable easier operation or other advantages. The computer and projector are not shown for clarity. This color-coding is programmable and can be done for various reasons and at various times, in various ways. In the particular case shown, the shaded controls (two knobs 910 and 911 and four switches 930 and 931 are colored the same color, for example red, as being part of a family of controls used to adjust let us say the sound quality of the audio system. Other combinations of colors can be chosen for different controls and functions. The color designation can even, if desired, be user selected via program choices on a pc, and downloaded to the vehicle for example. Since many of the controls may be programmed to serve multiple purposes (for example an Audio Volume knob one moment, and a Heater Temperature knob at anther time) such color-coding is really very useful. For example, the knobs 910 and 911 when used for a Climate control function might be coded yellow, along with say three but not all four of the switches 920 and 923. Indeed the other switch if not used at all, might have black projected (ie no light) such that it became much less visible in the Climate mode.

For those operators such as the inventor who are red-green color blind, it is possible through just a program change to provide them with a meaningful color palette they can see, for examples bright blues and yellows rather than the typical green and red often chosen.

In addition or as alternative to color coding, it is also possible to programmably can turn on and off various illumination features to a device. For example, when first put in radio mode, one can choose to have only radio related buttons light up on the center stack, making it easy to see which ones mattered to the instant task at hand. The other buttons could be dark, or at lower illumination in this case. And the desired buttons may be color coded too. When the task was finished (noted by the movement to another control function for example) or after a time period had elapsed, the display can be programmed to simply revert to a standard color and display mode.

In addition, one can have different areas displayed. For example using the invention, one could black out those areas of the screen not needed for example to tune the radio. Or one may display a box around those controls needed, or cause the region to be shaded or any other technique making it easier for the driver or operator to see what to do.

For example too, one can push a button to work the bass controls of the audio system then use knob to control a big display of bass values (eg +2). Or could use three position button to go up or down. Functionally too, If a knob is changed in its function to a radio volume control, then all the switches might in one mode, turn to functions relating to that control

One may build switches according to the invention which also allow projection of data in their center portions. To make the switch work optically, one can detect a specified movement of the switch (e.g. a one mm movement in the plane of the screen) relative to previous position, or a fixed home position. Alternatively, one can upon moving the switch expose or cover up a reflector or other light indication which condition is detected, for example by a TV camera. This effectively determines that amount of light above a preset amount is present (or not present) in a given location, indicative of switch condition. this condition is due to a detected light increase due to the exposure of a reflector to light (or other light source) by the action of moving the switch.

FIG. 10 illustrates another center stack embodiment in which three projector/sensor units 1005, 1010 and 1015 in an over/under arrangement are used to maximize brightness and minimize depth in a dramatic large area center stack screen/control surface 1000. Each covers a relatively smaller area in the vertical direction, allowing more brightness and less depth, which further allows in many cases one to project directly without folding the beam path. In some cases, these projectors could via beam splitting arrangements, share the same laser sources if desired. Note that the displays can be stitched together in the computer to form a large contiguous display for example at point “A”, and/or trim strips such as 1030 can be used at the display junctures if desired (particularly if as shown at point “B” the two projections don't meet). Note that by having the fast axis of scan in the vertical direction for each projector, the speed of scan required in that direction can be less, as larger scan is required in the direction out of the paper (typically by a 16:9 ratio).

This particular embodiment illustrates a version useful particularly in small cars where the instruments and gages (speedometer, fuel gage, etc) are projected in the top region and shielded a hood from sunlight. In this instance, the top projector can be optionally replaced by a conventional LCD screen if no physical controls are needed in that location, and if desired the LCD display “stitched” with that of the projector below, as disclosed in my co pending application Ser. No. 12/715,097. Alternatively the projector could be replaced by a conventional touch screen flat panel display if one might wish conventional touch functions to be used.

It is also noted that the bottom projector of the three may not be needed on certain vehicles that have limited function to fulfill. Indeed because the invention can reconfigure a screen and control surface to perform both audio and HVAC functions, just one projector/sensor unit can be advantageous in low cost vehicles, similar for example to the double din example

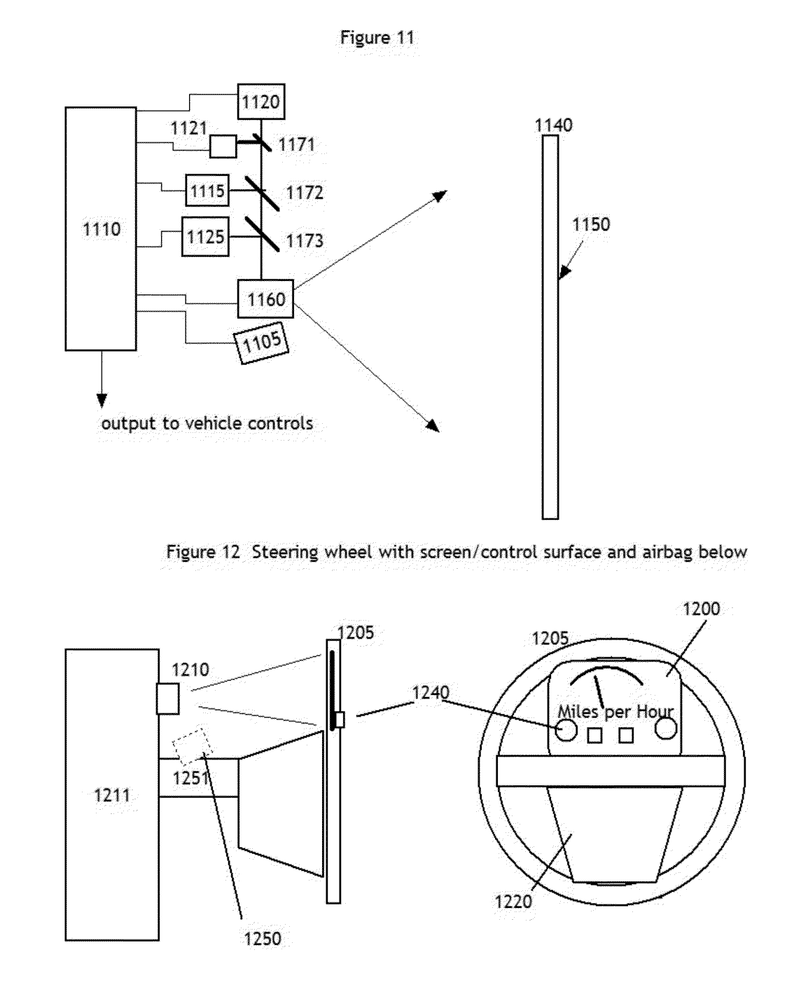

FIG. 11 illustrates a basic sensor/projector embodiment also containing features for improving brightness and general visibility of labels or other important information during conditions of sun irradiation of the screen/control surface. Computer 1110 controls both projection and sensing. The scanning projection mirrors 1160 scan the Red green and blue combination of lasers coming from unit 1125 which have been previously combined in side 1125 in the proper proportion for the pixel to be projected on to screen and control surface 1140. An IR laser 1120, operating at for example 880 nm is scanned simultaneously with the RGB signal (the later being turned off when nothing visible is to be seen). Detector 1121 (which may have a band pass filter to reject information not at the 880 nm IR laser wavelength) in the return path picks up information as to physical control positions on the screen and control surface. Dichroic minors and beam splitters 1171, 1172 and 1173 are used, for example, to combine and direct the beams.

Sunlight or other light 1150 hitting the screen and control surface is detected in this example by a separate detector 1105 responsive in the visible. The light detected is used to control in this case wither more power or time to be spent on labels and critical information, or to turn on auxiliary laser 1115 to provided added intensity when labels are projected.

As shown for example in my co-pending applications, it is often useful to maximize display brightness for critical functions. One may in certain cases preferentially increase light intensity of labels important to the drivers perception of the controls, while decreasing the intensity of other projected information, patterns and designs in order to keep the light sources (typically diode lasers or LED's) within their safe duty cycle. With scanning laser projectors this can be done by causing the scan beam to dwell longer on the control labels (e.g., the word TEMP in the center of a knob being used to control heater temperature) and shorter on the non-essential information, which could be even decorative in nature. Alternatively or in addition to changing the dwell time, one can briefly overdrive the light source when writing the labels (for example with a momentary high current), making up for this increase if needed by under driving the source when not on a label. This has the advantage of being able to use constant scanning velocities of the mirrors or other optics used to sweep the beam.

Additionally, or alternatively, it is also possible to use one laser (or even an extra laser), for example a red laser which is widely available in high power levels, which has considerably more power than needed to cooperate with the green and blue lasers for the RGB color generation. This laser would be gunned up in power when needed to illuminate labels, shifting the color of the labels toward or to, that laser color. Green would perhaps be best due to the eyes higher sensitivity, assuming green laser prices are acceptable, which they are not today.

It is also possible to change the size of the label, either the size of the letters, or their thickness or both, in order to improve visibility. This also applies to those persons having difficulty focusing on the screen when the labels are smaller. To facilitate a major change in size, it can be desirable to abbreviate the label, possibly with a pictograph if such is clearly understood.

FIG. 12 illustrates an embodiment with screen/control surface 1200 in the steering wheel of a vehicle 1205 with the projector/sensor module 1210 in the instrument panel 1211 ahead of the wheel. This arrangement still allows use of an airbag 1220 below the screen and control surface, with little or no change from present airbag in steering wheel designs. Projection is fixed, even though the screen/control surface rotates with the wheel. (or it could even remain fixed if it was just behind the wheel and anchored to the steering column rather than the wheel). As the angle of wheel rotation increases, this can be sensed by the projector sensor unit and image projection can be reduced in size and altered in shape where desired to fit on the screen that is in view. It should also be noted that the sensing of control position (such as that of knob 1201) and finger touch position, needs to be invariant to wheel rotation if one wishes to have these functions operate with a turned wheel. In this case, the reference points for the controls can be taken from a reference such as mark 1240 on the control surface 1200, which is tracked with wheel rotation and can be used to provide same for use in compensation of sensing and projection.

It should be noted that the screen/control surface 1200 in the wheel as here disclosed, may block any instrument cluster on the Instrument panel (when it is planned to be located in front of the driver). For this reason, the projector may optionally provide information commonly provided in the cluster, such as speed, RPM, fuel gage, odometer and the like.

As an alternative to the instrument panel location, the projector/sensor module (1250, dotted lines) can be located in the steering column 1251. It should be noted that the screen/control surface 1200 does not need to cover the whole wheel radius from center to the periphery, and if the screen and control surface is located just near the center, it is also possible to see conventional instruments located on the instrument panel. Naturally one application of the invention however is to replace these with projected instruments on screen 1200, as well as to provide other useful information such as night vision images, and allow reconfigurable controls to be used by the driver in the steering wheel.

The projector/sensor unit in this case may be for example located in the instrument panel directly in front of the screen on the wheel, or in the steering column. To accommodate the airbag used in the wheel, the screen can be above the airbag as shown, or the technique of a co pending application used wherein at least a portion of the screen is in front of the airbag, with the projection coming in from an angle such that the projector/sensor unit is out of the way of the airbag. In this case the airbag blows through the screen and control surface upon detonation.

FIG. 13 illustrates a front projected sun visor based embodiment of the invention, wherein the projector/sensor unit 1300 (connected to a computer unit not shown) is roof mounted to vehicle roof 1305. A sun visor in its stored state 1311 when not used for image presentation, control, or sun blocking purposes, is swung over into position 1310 and used for display and control. When desired for blocking sunlight fully coming through windshield 1306, a slidable portion 1325 of the visor is pulled down from within the visor to increase its vertical height in the downward direction.

Light from the projector is projected and reflects from the surface of visor 1310 facing the driver 1330. The angle of the visor when swung down from the rest position, is fairly constant, such that with suitable design of the reflective elements of the screen 311 on the back of the visor the projection can be preferentially reflected with high gain back to the region of the drivers eyes. The projector may not be directly over the driver's head, but displaced toward the center of the vehicle so as not to be in the way.

As shown, the device has several purposes and features. First, it should be noted that the visor in a typical vehicle when pushed past the vertical toward the windshield, is quite visible to the driver, without blocking too much of his forward vision. This means that it may be desirably be used for both image display and control purposes using the invention.

In the first case it may be operated in an image generation mode, for example, where an image of for example a night vision image is presented, as an alternative to having such presentation in a heads up display on the windshield, or in an instrument cluster. However, the advantage of this visor location is that the visor is accessible by hand by the driver which also means it can be used for reconfigurable control purposes. This is not easily and intuitively possible with either HUDS (Heads up displays) or conventional instrument clusters behind the wheel.

Note that one can locate a screen and control surface using either front or rear projection, in the portion of the roof 1370 in front of which the visor in its parked state 1311 resides. In this case, one just need swing the visor away from the zone 1370 in order to use the controls there.

Note that one may also arrange the system such that the sun visor, in its stored location 1311, may also be used by the driver for viewing of displayed information thereon as well as for control of vehicle functions using the invention. This has the advantage that the reflective bead or other screen material is on the back of the visor when rotated down, leaving the present vanity mirror as it is now. However the angle of the visor in this stored location is typically parallel to the roof at that location and thus very oblique to the driver and to a roof-mounted projector. This can be helped a bit by making the hinge locations stick out from the roof about an inch or so, allowing the front of the visor to be a bit downward from the roof. And it can be helped by spacing the projector away from the roof, which is easier to do if the projector is toward the center of the vehicle where it does not occupy headroom of the persons in the left and right seats.

When the knob or other control is on the part of the visor facing the driver when swung down, it is useful to provide a recess in the roof headliner for the knob to fit into.

An interesting aspect of the invention in its ability to optimize light energy from the projector/sensor unit seen by the driver or another selected recipient.

FIG. 14 illustrates an embodiment of the invention in which a screen/control surface 1405 may be temporarily pulled down in front of the inside center mounted rear view minor 1401 of a vehicle, in order to act as a control device, or a image display (for example of video data taken of, or from, a trailer one is pulling). In this example a roof mounted projector sensor unit 1410 illuminates the surface 1405 via front projection, but a rear projection arrangement may also be employed. In either case high gain is possible as the drivers location is relatively fixed, other than the variance in size of drivers. A control device such as knob 1430 is shown.

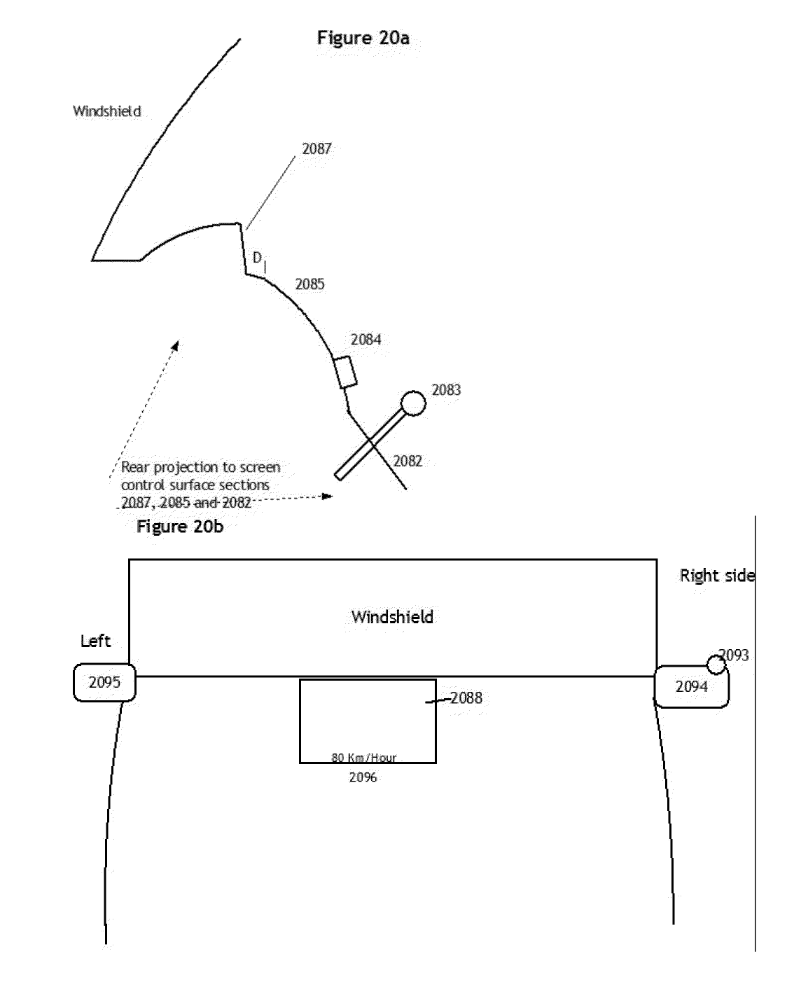

In those embodiments in which the display is for use by the driver, and is in the sun visor or steering wheel, or in front of the mirror, I feel that these may become viable for control, generally only if there are physical, intuitive and familiar controls such as knobs sliders and switches on the screen/control surface. A pure touch screen type function, while interesting and flexible, lacks traditional feel and understanding. Thus physical control configurations disclosed herein will make systems in unusual places like the visor or steering wheel viable. If it isn't totally intuitive, it may be dangerous. It is also noted that a screen suitable for projection in this manner may also be built into the rear view mirror and used for same, when vision to the rear is not needed, or may use input from a camera pointing to the rear to provide such vision. See also FIG. 20.

FIG. 15a illustrates the instrument panel center stack of the invention contrasted with a conventional vehicle stack provided in the same physical space. Compared are an RTD Automobile Instrument panel of the invention disclosed in co-pending applications to that of a typical mid line or low line vehicle. Particularly emphasized are advantages for elderly drivers and others with disabilities.

For illustration, some examples of screens/control surfaces of a conventional and an RTD instrument panel are diagrammatically provided in FIG. 15a. Represented is what's called the “Center Stack” in the middle of the instrument panel between the driver and the passenger. Today, this region typically houses the audio system (Radio, CD, etc), the Climate control system (also called HVAC, for Heating Ventilation and Air conditioning), and various types of switches for miscellaneous functions. In high line vehicles such as a MB S-500 for example, there are still more switches and a navigational display to cram into this space as well. I believe this causes substantial driver distraction in operation thereof—especially for the elderly. However, some alternatives to this approach, such as BMW's i-Drive, appear to be even more confusing, at least to some drivers.

A fairly typical center stack of 10 inches wide and 13 inches high is shown in FIG. 1a, together with an RTD version of the invention in which the whole 10×13 surface is a display with controls on its face. The display and virtual controls area may all become a rear view display on backing up. It should be noted that the whole area for example 10″×13″ (25 cm×33 cm) RTD surface may be a display area, and can be configured accordingly. It also may be operated as a randomly accessible touch screen. This allows intuitive interaction with displayed data of any sort, including data and video images. Some unique ways are employed to re-arrange vent location to free up display space. In the FIG. 15a example, selection of which screen/control surface function to work with is made with a large physical knob labeled selector in this case (one can programmably change the function and label of the knob according to the invention).

FIG. 15b shows the Audio section of the instant invention with function selection via lighted virtual icon buttons running down the left side. In one exemplary case, these buttons when touched, register a signal that the one touched has been selected (i.e., a touch screen type function). For tactile feel reasons, it is noted that selector buttons are however, often preferably physical switches rather than virtual via a touch screen type function.

The RTD version of the invention is shown on the left, which is contrasted with a typical conventional arrangement of today depicted on the right side of the drawing. The RTD audio section uses in this example the classical “Radio” metaphor having two knobs on each side (don't forget to show possible tactile ridges on the fader etc). Radio preset buttons are virtual in the usual place below the knobs. It should be noted that the CD slot for the CD's or other media at the bottom can have labels or other data such as the artist displayed in large letters near it when the CD player is in use. Other media such as MP3s and the like can be labeled and playlists also displayed Alternatively this data can be at the top of the screen or any other desired place the manufacturer wishes to put it (or it may be a feature provided for the customer, that he can chose the location desired). The data can be projected on the screen per these instructions by suitably programming the display control computer.

The HVAC section of the RTD instrument panel in this case uses the same two knobs as used in the Audio section, but reconfigured in this example for temperature and fan speed. The radio preset buttons have been programmed to become, via a different video projector program, the air distribution selection buttons in this HVAC reconfiguration. More or less than 5 buttons can be used, by simple reprogramming of the unit, both in the display of button locations and in the sensing of touch to correspond to those locations

FIG. 15c shows the RTD Audio section reconfigured to HVAC (climate) by pressing one of the illuminated selection buttons, in this case the Climate button. (which also can have a unique feel to it, by imparting a acoustic wave feedback to it for example as described in co-pending applications).

The conventional center stack illustration above is similar to that found in many vehicles often purchased by older buyers. This is a plain looking, planar vertical stack curved slightly on the sides as shown in FIG. 15a. While representative of many minivans and some SUVs, it does not represent style trends toward instrument panels curving away from the passengers (to provide a feeling of spaciousness in the passenger compartment), nor is it representative of the clutter in some other Instrument panels, which have more electronic equipment to accommodate. The conventional center stack typically includes:
Audio section with standard radio and CD or other media player
Climate control or HVAC section (heating ventilation and air conditioning) of a standard unit, without extra controls for right side passengers or automatic temperature controls
Switch section for optional features such as traction control. This is located at the bottom and most out of the driver's line of sight, as these switches are least used.

On the conventional vehicle center stack illustrated there is no LCD flat panel display nor other display for navigation or other purposes, and unless everything was redesigned, there is no room for any such display as well.

In addition to the controls of the center stack, there are also in many vehicles (typically those of GM) two stalks on the steering wheel which control wipers (various functions), cruise control, turn signals and in some cases lights. These stalk based controls are expensive, trouble prone, and occasionally cause problems since actuation of one function can inadvertently cause another to switch.

There are also other switch controls on the instrument panel and in the interior for headlights, interior lights, seat adjustments, power windows and locks. And this is just on a standard vehicle today. Fancier vehicles have more functions to justify the higher prices asked.

Even in this simple conventional example:
Some controls are too small to read without glasses (for anyone far sighted driving the vehicle). For example the words push on the radio of a Buick Rendevous brand vehicle are in letters one mm. high-virtually un-readable by an elderly driver while driving.
Even for younger drivers, many labels are of such small size that they require refocusing of the eyes to see, from far to near (for those whose age allows them to do this). This is not desirable while driving if it can be avoided.
Some labels are not intelligible as to meaning
Other controls are provided for functions not provided, wasting space and adding to the confusion. This is particularly on the radio section
Some controls of the radio or heater require scrolling which requires too much concentration.

As one example, on a radio of a 2003 Buick Rendevous (a typical vehicle of this type) a knob is named both Audio as well as Tune. You have to read the illegibly small letters “push” to understand how to change from one to the other. And you have to know from reading the manual that once doing that you may get to the fader section, by scrolling thru one by one. All this takes knowledge and is not at all intuitive. And the process of scrolling thru menus, like in other vehicles that require this, takes needless time from driving duties. In one example, the owner of aforementioned Rendezvous absolutely could not operate it while driving. His wife tried and couldn't either, even though she was in the passenger seat. As a result, their daughter's request to have the music play in the back and not the front couldn't be met. This is very frustrating and illustrative of a non-intuitive system. And this is one of the simplest!

It should be noted that with the RTD invention, visual display of TV Video and/or virtual controls on the top part of the display surface may be provided (also a touch screen in preferred embodiments), which is now freed up due to the multifunction nature of the Audio, “HVAC, and Switch functions. Where vents are present in the center stack, the display, if desired, can “wrap around” the vent shape in the area generally occupied by the display region, creating more space for display or data functions. However, the display surface can also be used to account for added conventional functions in periods where normal regions are overloaded, or where it is desired, for example in stress type situations, to have control functions in the region at the top most in the drivers line of sight.

FIG. 15d illustrates a blank control and display section 1500 with nothing projected (or alternatively a display of some background or pictures of interest to the driver such as family and pets). In this case the two fixed physical knobs 1505 and 1506 are shown, which can be functional for some purposes even with a blank screen/control surface such as 1500. As noted above, functions may be for example, be selected using the row of virtual buttons (which can alternatively be physical buttons) arrayed, for example, along the left side of the device (as in FIG. 15e), or, as shown here, by a selector knob 1501, or any other suitable means.

The above also serves to illustrate the space available for information display, when a traffic alert comes thorough, necessitating either picture information or text or both to be displayed as big as possible. In this case the total surface shown, less the space occupied by the physical knobs, is available. When the alert has been dealt with, the operator of the vehicle can signal the system, for example by pushing on virtual switch “A” in dotted lines, at which point the system can revert back to whatever setting it was in. Alternatively, reversion can occur after a suitable reading and understanding time period, say 20 seconds.

FIG. 15e illustrates the provision of switches to activate different functions replacing the switches often conventionally provided on instrument panels today in a row at the bottom. By selective reconfiguration, these switches too can take on added meaning, and allow other controls to be eliminated, saving cost and complexity. For example, as shown, one could have controls for Wipers, Cruise control, Power Seats, Power Windows and Locks all in the center stack as shown. In this case illustrated, the Radio is at the moment not presently displayed or activated, but the knobs for it are assigned to other functions relating to the switches, in this case the wiper and lights controls.

FIG. 15f illustrates an alternative to FIG. 15e where the radio (audio) function is still present, along with the heater (Climate) in virtual form. However, with much reduced size of labeling and associated radio information when the climate control and/or switches are being used (all choices could be user or factory set).

Co-pending applications have disclosed the unique ability of the invention to allow the lettering for any given control function of the moment, to be maximized in size, and placed optimally, in location, and orientation for driver comprehension, even in different driving situations. And the whole format of the screen/control surface display can be so adjusted as well

FIG. 15g illustrates a change in size, position and in some cases arrangement of instrument panel information when a function is activated. This can be as simple as sensing that a knob, say the volume knob, in the radio arrangement of FIG. 15f above, is being turned, and as it is, expanding the size of the lettering for that knob or for the radio complete to allow easier viewing, as shown in the before turning and after turning examples. Thus even if the labeling had to be small to accommodate the other switch functions, it can immediately become visible when needed.

While shown in the figure relating to markings on physical controls such as knobs, it is also possible to increase the size of touch screen related functions, such as label size color or shape, icon box size or shape or color, or graduations. One example would be the touch function of the audio equalizer below, which when one slider bar is touched can expand the definition for example of that bar.

Alternatively, the control functions, instead of becoming small to make room for the expanded radio display, expand to occupy the region of the video image and data display at the top. In this case, the controls are virtually depicted sliders for the equalizer audio function, but could be anything else desired. They can be programmed to revert back to a previously displayed set of information, for example after a given time period or when radio activity ceases, if desired.

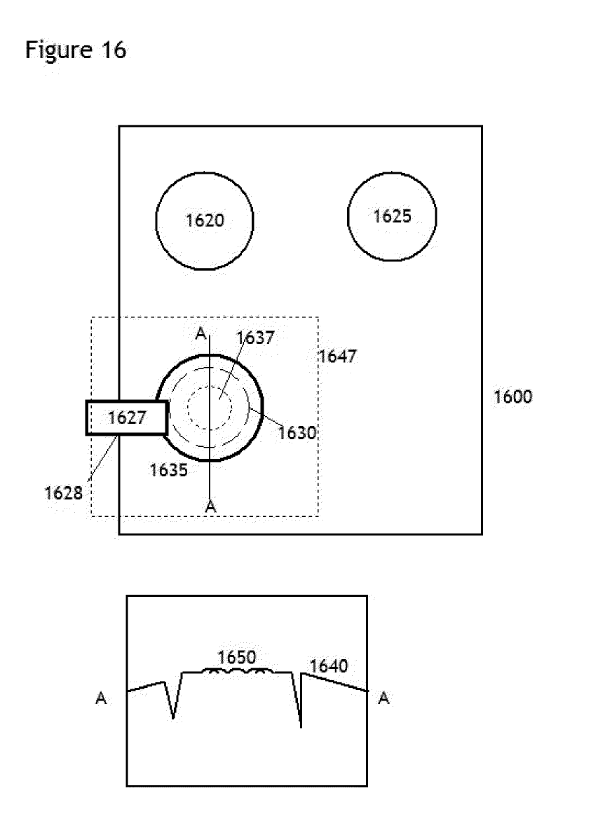

Following from FIG. 3, FIG. 16 further illustrates sensing techniques, social and safety issues associated with assisting persons in their homes. Consider the image 1600 of the stove top and its environs captured by camera 1601 such as a CMOS webcam made by Logitech Corp. having 1.3 million pixels. This camera can acquire the image at least 30 times a second assuming sufficient illumination, which can be provided as described by LEDs if room lighting is not sufficient.

In a first application example, the camera is used for determining the state of boiling in a pan on one or more range burners whose images are 1620, 1625 and 1630 shown in dotted lines, as covered by pan 1635. If the stove control and camera are controlled by a common computer system, the camera may be used to look at the image region of a burner when that burner is on. A pan image thereon is detected and this indicates it may need to be monitored for boiling conditions. Also sensed (and connected to an alarm function, such as audibly through loudspeaker) is a condition that the burner is on, but no pan is on top of it. This is undesirable in general, and certainly if too much time has passed.

The second thing sensed is the water or other liquid in the pan, and its state of boiling. This can be determined by looking at the image region 1637 in the central area of the burner where all pans are placed such as 1650 and analyzing the effect of bubbles in the water on the image. The more high spatial frequency change in the image, the more bubbles and the higher the state of boiling. For example, image trace 1640 of a line AA across the image of a central region of a camera field region of interest (ROI) 1647 on burner 1630 where a pan image 1635 is present has high frequency noise 1650 on it indicative of boiling, whereas image trace 1645 does not. Such change can alternatively or in addition be analyzed in a time based manner by continually subtracting images of the pan liquid in the region

The simplest action in the boiling water monitor is to sound a audio or visual alarm that the water is boiling. However, when automatically connected to the range, it can actually turn the heat down in various manners, for example to decrease to a low boiling state, or even to turn off the burner entirely. Conversely if boiling is supposed to happen and doesn't, it can also signal an alarm.

It is noted too that the camera may have its own microcomputer associated with it, in order to process images and sound alarms, independently of the range controls. In this case the three burner regions are constantly looked at and a pan image determined, if any is present. This is dark ring on generally reflective background. If a pan image is there, boiling conditions are sensed in that pan, as one processing example.

The other principal use of the camera system is safety. The primary issues here are to sense for unusual conditions on the stove, such as persons entering the stove area, burners on but with no pan, and pans such as 1635 with handles dangerously sticking over the edge of the range, where they could be hit by small children.

The image of the region of interest of the stove top and a zone just outside it is captured. As shown also in the image 1600 the pan handle sticking over the edge can be determined by analysis of the image, where the handle image 1627 clearly bisects the image of the edge of the range 1628.

A static situation existing for more than a fraction of a minute such as the pan handle is easy to determine and generally separable from normal motion around the stove in food preparation activities. More difficult to determine without false alarms is if something enters or leaves the space which shouldn't be there. One method of simply dealing with this is to set an alarm when the cook leaves the area of the range. Then any object entering the periphery of the stove 1600 image can be treated as reason for alarm.

A second method also relatively simple, is to say that objects larger than a certain value can't be present for any significant length of time in the region of the stove. This could also exclude generally circular objects, such as pans. Objects larger than arms which are not round in nature could for example cause an alarm. A third method is to look for certain movements, such as stirring and exclude those from alarm. A fourth way is to look for color, particularly flesh color, using the color sensing ability of the camera. The exact way such an alarm would be set up generally depends on what is expected in the normal course of activity, how old children are in the home, and the habits of the cook. If all pans and the stove top were not flesh color, it makes it easier to discern the presence of a person near the stove and burners.

FIG. 17 illustrates a rear projection embodiment for stove control, and miscellaneous entertainment. In this example several novel aspects are illustrated, in this case relative to control and use of a stove and range 1720. The rear projection engine 1700 consisting of a LED projector with wide angle lens and an associated sensing camera and display and device control computer as taught in co-pending applications is up in or near the range hood 1701 (or alternative microwave device), in a cool area as I have taught in my co-pending applications for other purposes. On the rear projection screen portion 1710 is the controls, in this case familiar knobs and switches such as knob 1711. The controls can be physical controls (also made of heat resistant material, which can be opaque even if projection thru them is not needed. The screen is Pyrex or other transparent material capable of withstanding the heat from the burners and oven. Control can also be effected by touch or gesture as taught elsewhere and in co-pending applications. Internet or other data connection 1715 allows recipes, instructions and social interaction via full size images of famous chefs, movie stars or any other desired scenes, video or still. An optional camera 1740 to determine actions of the person as discussed below may be included as well.

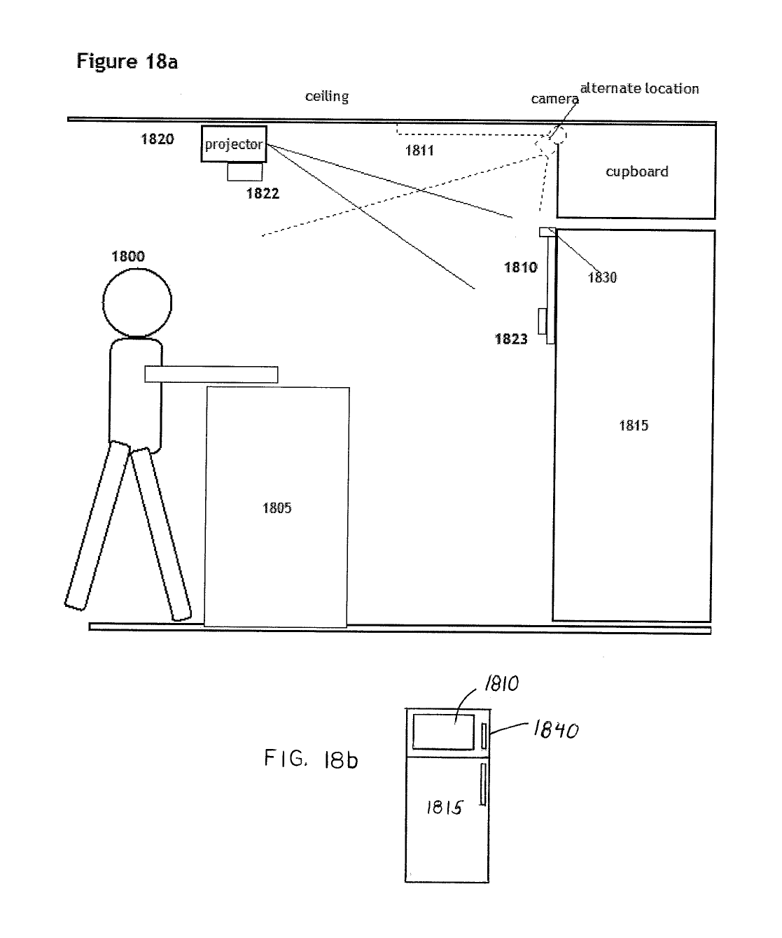

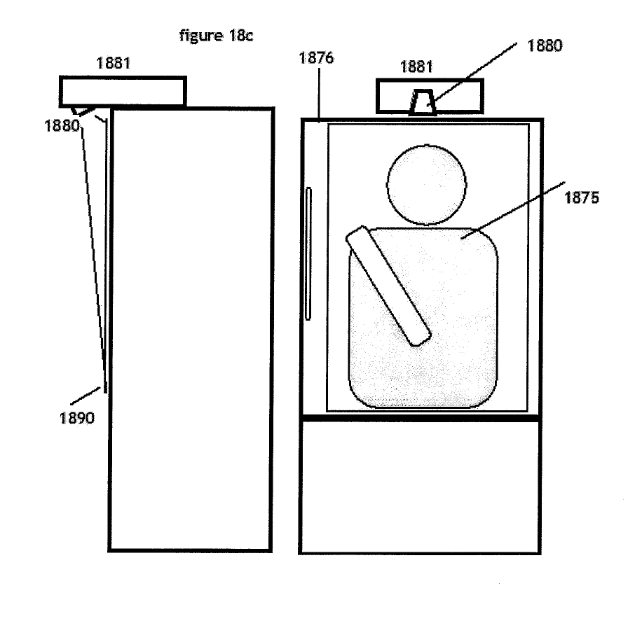

The invention can be further used to assist persons living at home in other ways. FIG. 18a is a front projection embodiment including sensing of characteristics or actions of the person or persons in a room, in this case the kitchen, in which a person 1800 is working at a island type counter 1805, and a screen 1810 displaying preferably life size images is provided on the top freezer portion of a fridge 1815.

The overhead video projector 1820 which can be used to project TV images, recipes from the internet and other information such as described elsewhere in this application. A camera 1822 attached in this case to the projector (or collocated with it) and a computer controlling the projector (not shown for clarity) senses a control such as knob 1812 on the face of fridge 1801.

Further illustrated is a camera 1830 looking outward at a person or persons in the kitchen. A head, hand or face gesture performed by person 1800 can be sensed by this camera, and after its image is processed to determine the gesture, can be used to control the TV image of overhead projector 1820. An alternate fold down screen 1811 is also illustrated, where it is not desired to have the screen on the fridge door. This screen can be above the fridge, or be a large version covering fridge and cupboard above.

In this embodiment gesture and voice recognition is used to communicate with a camera located in the range hood or in another location. In addition other characteristics of the person or persons in the kitchen can also be sensed and used as input to a program controlling the display, loudspeakers, appliances or other apparatus. Such characteristics can be the presence of a smile on their face, the identity of the person, the utensil such as a spatula or mixing bowl the person has in their hand and the like. However it is noted that typically the background is constant when viewed by camera 1822, or to a lesser degree when viewed by camera 1830. Thus a subtraction of images with and without the signaling item, can be done to make it easier to discern.

FIG. 18b is a front view facing the fridge showing the freezer section 1840 on top of fridge 1815, which is provided with screen 1810. This screen can be permanent or temporary and removable as desired.

FIG. 18c is another fridge embodiment shown in front and side view. In this case it employs a screen 1890 on the door 1876 of bottom freezer type fridge allowing a quasi-full length substantially lifelike image 1875 to be displayed on the fridge door using projector 1880 with optional electro optical sensor unit if desired) in mount 1881. While the projector could be ceiling mounted, in this case the projector is mounted to the top of the fridge, and projects obliquely onto the screen surface on the front of the fridge door. In some alternative cases such a projector could be side mounted or wall mounted, and obliquely projected sideways onto the screen.

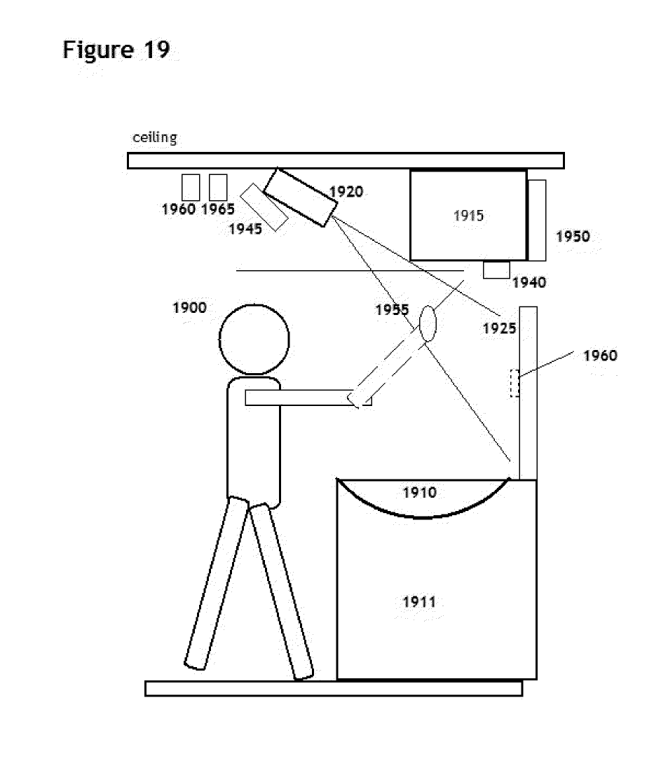

FIG. 19 now further illustrates human interaction in an embodiment employing a front projection display behind a sink in the kitchen, bath, laundry or other location at which a person may be working. As shown, person 1900 working at a kitchen sink 1910 in a counter 1911. Overhead there may or may not be a cupboard 1915, though there is generally at least a cupboard above and to one side or other. A projector 1920 projects information onto screen 1925 as in the embodiment of FIG. 18 above, and integral camera or other sensing capability can sense control locations on the screen if desired, for example to control associated equipment nearby such as a dishwasher, garbage disposal or trash compactor. Similarly too, data can be manipulated or displayed, and other activities in the home monitored or controlled as also described above.

Also illustrated are added control modes, and the unique social interaction aspect of the invention. For such control, a camera 1940 is provided attached to the cupboard as shown. Alternatively or in addition a camera 1945 can be associated with the projector as noted, which camera might also view the person as well as objects on the screen. The cameras and projector are connected to control computer 1950 with wires not shown, or wirelessly. The camera or cameras can be either a 2d or 3d type. Or a combination of the two. Auxiliary light sources such as IR Leds can be provided as desired to aid the detection of signals by either or both the cameras which may be employed.

An advantage as noted earlier is that the screen of the invention can operate successfully in this environment, without electrical hazard. The screen can be splashed without damage, and even take reasonable hits with pan handles, silverware, etc.

As noted above, it is a goal of the invention to enhance the social aspects of cooking and kitchen activity. Having life size images enhances the feeling of interaction with persons whose images are displayed, be they family members, movie stars, famous chefs or even pets. This interaction can as noted above be by gestures or voice (via microphone 1960 and voice recognition software), and the person whose image is displayed can in turn talk to the user via a loud speaker such as 1965. The images displayed can be live, via Skype TV for example to the computer 1950 driving projector 1920 and interfaced to loudspeaker, internet and microphone as desired.

Besides the sensing of physical control locations such as knob rotational positions, there are two main additional uses of the cameras of this embodiment. The first is to see human gestures or control objects, in order to cause some action, such as changing a tv channel of the projector, or controlling a stove to turn off a burner. For example, a simple case is if person 1900 wants to indicate a projected icon box 1960 on the screen 1925 (shown in dotted lines). In this example they just raise their hand 1955 until it blocks the projection to that box, which blocked condition is sensed by camera 1945 which senses the absence of the projected box. As long as the choices are relatively few such that obscuration doesn't happen, this works well. It is noted that if the hand 1955 is highly visible, that the hand itself, or an object in it can be sensed. But the person has no reference as to what that means, unless it is a movement gesture in space such as waving ones hand in an S motion say. This movement can be sensed by camera 1940 or camera 1945 for example. Given the problem doing that from a human interaction point of view, it seems better to just block things on the screen. A criteria can be the top most projected image graphic blocked is the answer, that is in the vertical direction of the drawing. This assumes the person is approaching from the bottom of the screen, which is actually (and helpfully) in the in-out or z direction due to the angle of projection in this instance. It would be less so, if the projector was projecting at a high oblique, like FIG. 18.

One can also use the camera 1945 or 1940 to observe objects the person might hold up to indicate a control function. This can be portions of the person, such as a head, hand or finger, or it can be something for example held in the hand. For example, if the computer recognizes that the person is waving a spoon, that might be a programmed indication to turn on the TV projection.

In addition to control activities, the camera and associated machine vision programs in computer 1950 can also be used to sense other things as well, for example the state of the person or persons in the kitchen (or other room, if the invention is employed there). Such states sensed could be if the person is smiling or frowning, or the like. If the person does an action, like nod their head, turn their head or blow a kiss, this may be sensed. A clothing object can be sensed, like a design on a dress, or the type of clothing, such as a rubber glove or apron.

For social interaction, The system can look at the person this way and a program probably in real time from the internet source, can comment on their dress or their attire, to see if a friend is present with them to and allow the system to be interactive by talking through a loudspeaker and excepting information from them through a microphone and a voice recognition program. The computer can be on site that can be Internet connected to something elsewhere as well. This capability allows one to have interactive video sessions while doing dishes, talking to someone who is either real (such as a family member whose image is projected on the screen) or in some canned or animated video experience or game.

Some sinks have a window over them. If a window (or mirror in the bathroom case) is present which it is desired to view out of, the screen may be moved out of the way, for example by folding it up, or sliding it down into a counter, even by motorization. It is also possible to provide a window or mirror, which may programmably diffuse light. The device if located over a bathroom sink, can be used not only for information, but to provide camera images of you for example from views to the back and side to aid various activities. Since most people don't have windows over their sink, the visual image displayed may be of outdoor scenes to simulate same. Such an image may be provided by an LCD or other display, not just a projector. But it needs to withstand the water environment and this is very risky for most flat panel displays today.

Another vehicle center stack embodiment is shown in FIG. 20a, which illustrates a surface being curved toward the windshield in region 2085 and then having a more vertical section 2087 indented a distance D. It may optionally also be tilted toward the driver an angle to the plane of the paper if desired. And in addition one can have a screen region 2082 in which the transmission shift lever 2083 projects through a slot, hole or other cut out in the screen with data as to its position (PRNDL for example) and possibly other variant information relating to transmission or chassis functions for example projected next to it. Knobs such as 2084 and other details can be located anywhere on the above surfaces as desired.

On region 2085 the controls such as Climate and Audio are projected and knobs and other controls used as described above. In section 2087 however, the variable focal distance afforded by the invention, allows the graphical representations of speedometer, fuel gage, and other instruments to be projected on surface 2087 aimed toward the driver. These readings vary with the variable in question. When backing up, the instruments are not needed, and instead a backup image may be projected in the same region 2087 (and/or 2085 as well). Also at other times, more important information can override the instrument and gage projection as well. This can particularly be when camera or other sensor data of immediate value with respect to safety needs to be viewed (as in dangerous traffic or passing manoeuvres), when malfunctions or other vehicle problems occur, or the like. Even in these conditions, one may not overwrite everything, but leave for example as small digital display of speed remaining on the display. (Almost all other gage functions are not time important and presumably do not need attention during other crisis periods).

In some cases the instrumentation display on surface 2088 (2085 in FIG. 20a) can be alternated with a display of either the rear image, or an image as shown in FIG. 20b taken from a camera 2093 looking rearward along the side of the car and mounted, for example, in the right side mirror mount 2094 (also replacing same if desired). This image 2092 can be quite large (e.g. 30 cm wide and 18 cm high and easier to see than looking at the normal right side mirror (in left hand drive cars). Such side mirrors today are generally convex to provide a bigger field of view, which however results in erroneous depth determination (which is why they say objects seem closer than they appear), and can cause considerable problems in viewing vehicles approaching from the right, and in the blind spot of the vehicle. This problem is obviated by the invention, which puts a very large display closer to the driver with a wider field of view, and no depth error. While advantageously done with the projector system of the invention, display surface 2088 could be provided by another form of display such as an LCD flat panel display. It is also possible to provide, in the case of rear images to project the instrumentation such as a speedometer reading temporarily overlaid on the displayed image 2092. For maximal image visibility, one can just project small instruments (e.g. a digital speedometer number such as 2096) on the image in an overlaid manner, perhaps in color for easier visibility.

It is further noted that if one has the functions of the right side mirror displayed with greater clarity and blind spot elimination on the RTD screen, this means that no adjustment is needed for this mirror. If one then makes the left side mirror 2095 a bit convex (as all right side ones now are) then This mirror too will allow a broader field to be seen, and perhaps require no adjustment for different drivers as well. The lack of adjustment has two big advantages. It saves cost, and it avoids the common problem of someone driving off without effecting mirror adjustment (And thus not being able to see properly).

Another aspect is that the display of rear vision on the RTD screen (or for that matter another screen, for example an LCD type directly ahead of the steering wheel where the instrument cluster is in most cars today) can be automatically as well as manually controlled. As noted above, when putting the car in reverse, a rear view image can be shown-in the simplest case from a camera in the middle of the car facing rearward, preferably facing down at an angle to the horizontal so as to see objects near the bumper on the ground. Provision of this feature would materially aid in preventing deaths of small children who are backed over by their parents in their own driveways. It is my understanding that over 2000 such deaths occur in the USA each year. Here again, the fact that the RTD allows the biggest possible display on the instrument panel is a material aid in assisting a driver, for example in a hurry to get to work, or pick up another child, in seeing what danger lies behind. This is especially valuable in Minivans or SUVS with poor intrinsic rear visibility.

Another example of automatically changing the display is when one is in motion, and one swerves the vehicle, for example to pass another car. In this case a motion sensor can detect this action and cause the display to provide a right side mirror view, which completely makes visible the right side blind spot. Or it could display the center view. Or on the RTD big screen it could display both, one next to the other (At a price of reduced size for each—but still bigger than anything displayed today).

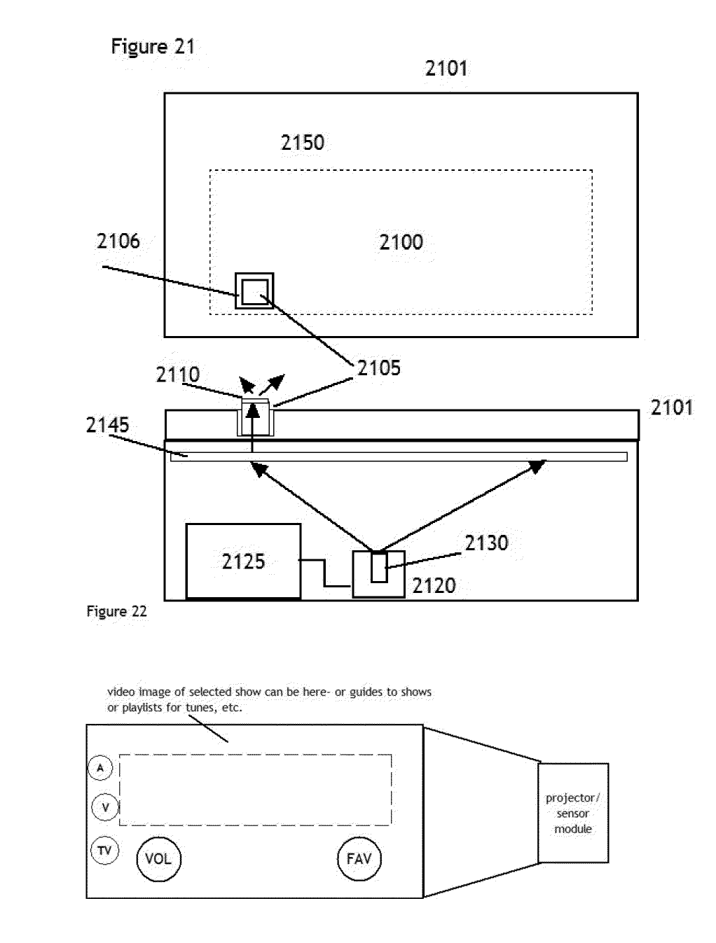

FIG. 21 illustrates a portable embodiment of the invention for use as a keyboard 2100, employing in this case keys such as 2105 (only this one key shown for clarity) which have a diffusing face 2110 such that with suitable projection by projector 2120 onto the face from the rear that they can change their labels under control of computer 2125. These keys can slide up and down in a plastic sleeve 2106 of square cross section as in a conventional keyboard, and can even make use of conventional means to determine they have been pressed. Or the state of being pressed (or even the degree depressed) can be determined electro optically, as in many of my co pending cases. For example using TV camera 2130 whose image is processed by computer 2125 in order to determine reflection changes from a point in the camera field corresponding to a key location due to the key at that location being pressed. Alternatively to a TV camera, a combined sensor/projector can be used, as disclosed elsewhere herein. Where keys slide a significant distance it can be desirable to use Fresnel lens 2145 to substantially collimate light from the projector.

Besides key information such as the letter of the key (e.g. “A”), the screen and control surface 2101 (of which the keyboard is a part) can have other projected image information in regions of its surface as desired, such as region 2150. This can be graphics, function keys etc. And given the invention herein these regions can operate as a touch screen. Indeed the keys themselves can operate this way, but the tactile feel is not what people are accustomed to. Keys alternatively can be comprised of deflecting membranes, which do have some tactile feel.

FIG. 22 illustrates another portable embodiment used as a remote control for a Video/audio system in a home (and optionally providing a degree of video and audio capability itself via its display and optional speakers).

This can be operated in a pure touch screen mode, but desirably for many people has some degree of conventional controls, such as reconfigurable volume knob shown, which also can act as a channel selector to go to ones favorite channels. Note that the remote itself has a screen that then acts as monitor to check out other station selections, rather than have the picture in picture approach, which can disturb others. One can also have insert-able portions comprising alternative screen/control surfaces such as a keyboard shown in dotted lines. This insert can be snapped to the back of the device and used as needed, or slid into place for example.

As shown there are two knobs, which when in normal TV watching mode represent the controls used most often such as a Volume knob, and a Channel knob to turn thru ones favorite channels-usually a set of say 10, but programmable of course. The beauty of the knob for this, like the knob of channel selectors of TVs long ago is that you can see it and turn to it immediately. The channel markings can be projected right on the screen next to the knob, or in the center of the knob.

Then when one wants an audio system, you press the hot button for that (labeled audio) and the Channel knob turns into a selector for play lists displayed on the screen. Possibly other functions would be needed, which can be provided as disclosed in other applications. And the same holds true for stored video files, accessed by pressing the V button shown. A pair of built in speakers (not shown for clarity) can make the entertainment coming from this portable control device complete.

Many believe that the remote control unit in a hospital room is the dirtiest thing there is in the room. One approach to solve this problem has been shown in FIG. 17 of my co-pending application Ser. No. 12/748,666 that allows the whole control interface to be sterilized or otherwise cleaned. Another is shown here, where the touch screen and control surface 2250 may be a separate member from the screen member 2260 illuminated by sensor/projector 2265 (and associated computer not shown, together with any wireless transmission devices such as IR, Bluetooth or the like) and this user interface member 2250 can be easily removed and washed along with the control details if used.

In a similar vein a device like that of FIG. 22 can be used as a Nurses notebook where she can document patient issues, and use the ability of the computer to call up images on the screen etc. Like many other applications of the invention this is usable in a high stress environment where menu based devices such as PDA's or Tablet PCs are hard to operate (and hard to clean).

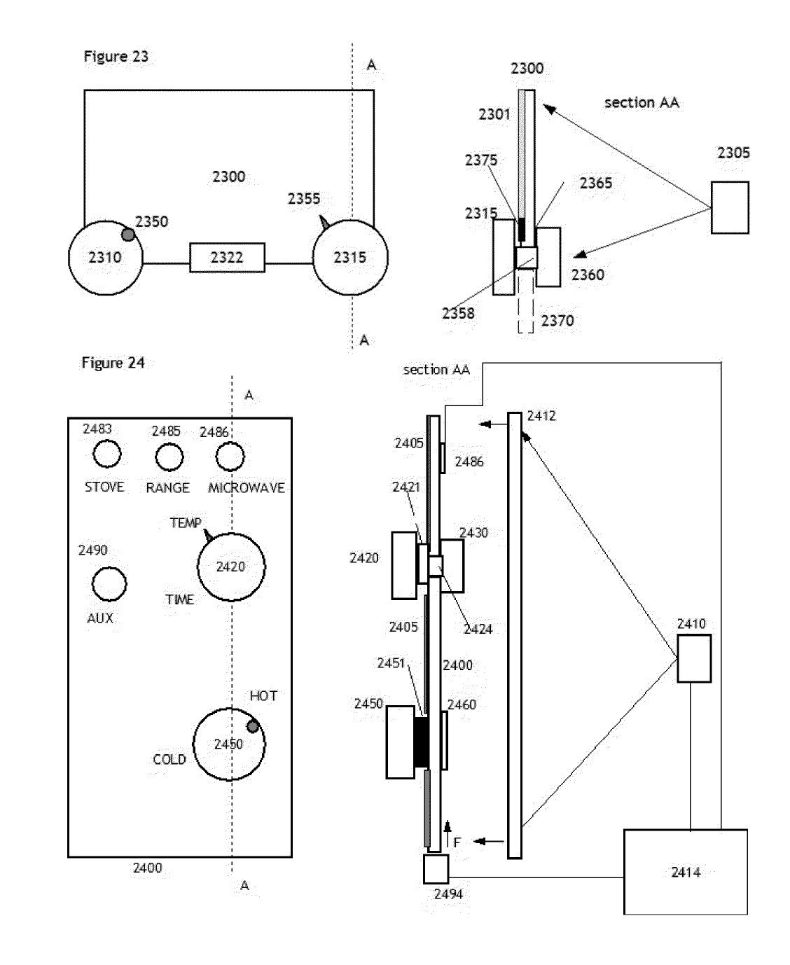

FIG. 23 illustrates a projector based control panel 2300 of the invention containing a diffusing screen portion 2301 suitable for automobile center stack or appliance, or other application having conventional knobs and switches, laid out similarly to some embodiments of my co pending applications. The screen and control surface 2300 is shaped to envelop a substantial portion of the two knobs 2310 and 2315 shown, as well as the pushbutton switch bank 2322. In this manner the labels and other information can be changed programmably with respect to said knobs and switches, while the design of the latter is conventional. Note the use of mask member in order to block some projection light and provide a uniform annular region around the knob. The surface 2300 may also be curvilinear over at least a portion of its surface as pointed out elsewhere and in co-pending applications.

The knob 2315 is turns a shaft 2358 of a rotary encoder 2360. this encoder is attached by adhesive 2365 to member 2300 and/or alternative support member 2370 (dotted lines). The readout and signal conditioning connections to the encoder can be provided in any way not obscuring the projection of light from 2305 to screen 2301.

An indicator portion of the knob may printed on the knob face such as dot 2350, or it may extend as in dotted lines 2355 to overlap the display front surface as shown, to form a pointer at any data projected on the screen such a label or graphic. Alternatively the pointer may be virtually projected onto the screen, to correspond to detected knob circumferential location. As another alternative, it may be projected onto the knob face itself, for those embodiments in which the knob has a display member, such as a projection screen on its face. It is generally desirable to use a knob potentiometer or encoder readout that is a shallow as practicable so as to not obscure data on the screen—especially If the knob or switch is not located at the extremes of the projector angles as shown in the side view of section AA. In some cases it may be desirable to have a mask 2380 such that stray projection light or diffuser effects to not make a displeasing image around the knob. Alternatively or in addition, one can use a Fresnel lens as shown in co-pending applications to substantially collimate projector image for projection past the knobs and switches (or other physical controls) and onto the screen. This will now be illustrated together with two other knob mounting methods

An alternative automotive center stack arrangement is shown in FIG. 24, in which case two knobs are substantially wholly within the confines of the control surface member, which has simply been cut out to allow them to be in place. A control surface member 2400 provided with diffuser 2405 is projected on by projector 2410 controlled by computer 2014 whose diverging output is collimated by fresnel lens 2412. The member 2400 has on it mounted two knobs 2420 and 2450. In one case a hole is provided in the member 2400 which allows the knob 2420 to be attached using a threaded nut 2421 which threads onto shaft housing 2424 attached to rotary encoder 2430. In the other case, knob 2450 rotates on an encoding member 2451 mounted to the front of the member 2400, and thus does not require a hole to be made therein. However this generally requires the wires to the encoder to be either transparent conductors or hidden in some way under trim mouldings or the like. An optional opaque mask 2460 may be employed to block projected light from being seen. The electrical connections of knob rotational position sensors 2430 and 2451 to computer 2414 are not shown for clarity.

In the example of FIG. 24, three switches, in this case non-contact types such as capacitive switches 2483, 2485 and 2486 sensing finger proximity are provided to switch the function of the control panel from one appliance to another, under the control of computer 2014. These switches are operated using electronic circuit connections, for example with transparent conductors on member 2400. The capacitive (or other types) of switches can be changed in their function just as can the function of the knobs. For example, consider the use of auxiliary switch 2490 which is used to change the function of the three switches from the appliance selection state, to a selection device for other functions, for example TV, Audio System, and Internet Camera interaction. In these new states, the function of one or both of the knobs can also be changed. For example knob 2450 can become a tuner knob for a TV station selection, while knob 2420 could be in this example permit playlist selection scrolling of audio files remotely stored. In both cases it is assumed that suitable interface and communication facilities are provided.

If an off axis Fresnel lens is used, light from the projector may approach at an oblique angle to the control surface thus decreasing the depth of the device. See also my co-pending applications. Note that a pulsing member such as solenoid 2494 can be pulsed to send a force signal F into the member 2400 under command of computer 2414 to indicate various actions or states of the switches or knobs shown, as disclosed in co-pending applications While the knobs switches or other units can be generally mounted to the control surface, they do not have to be.

It should be noted that the screens of the invention herein, particularly those in the vertical plane such as shown in FIG. 18, can be used for aiding exercise and rehabilitation as disclosed in my co-pending application Ser. No. 12/358,404. Simple and low cost sensing techniques disclosed therein using webcams or other single cameras for input of video game commands may be used also in this case for input of commands to control various home functions.

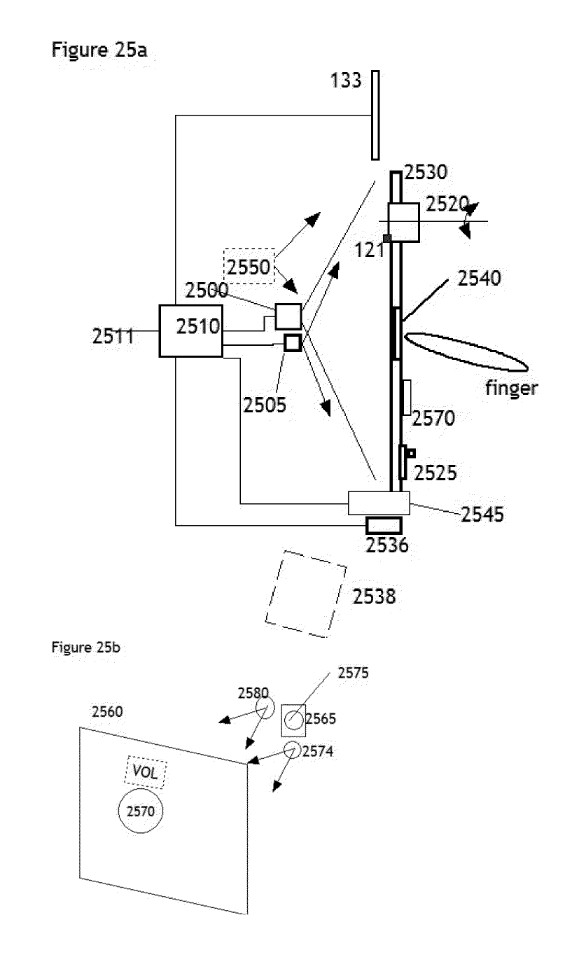

FIG. 25a illustrates a low cost control panel arrangement of the invention, which in the simplest case is used only for control, with little or no sophisticated display capability, It is ideal for appliances, trucks, cars and many other applications not requiring sophisticated display interaction. The central premise, also disclosed in co pending applications, is that a single low cost camera 2500 such as used for cell phone or automotive applications, together with a LED 2505 can be used to illuminate and sense the position of a variety and a plurality of physical control details such as knob 2520 and slide switch 2525 on control surface/fascia 2530. Image data collected by the camera is processed by simple computer means 2510, (running suitable machine vision software such as MIL by Matrox company of Montreal Quebec) and outputs such as 2511 provided to I/O used to effect the control selection made by the user interacting with the knobs and switches (and other controls such as dials sliders, etc as desired)

This system is very inexpensive, and the camera and its processing portion is as little a few dollars in large quantities, and the led and the power supply may only be a dollar. More expense is needed for general computer and I/O activities, common to all control panels.

Retro-reflectors or other high contrast targets may be used on the control details such that little light is required for their illumination. The control here is not exposed to ambient light so no need for higher powers or sophisticated computer processing. Nor is there a need for near IR operation or band pass filters. These can however, be used if some ambient light issues persist such as where light can leak into the housing via windows used for tell tale illumination or labels (see below)

Not only is this aspect of the invention inexpensive (on the order of say 30 cents per controlled variable or device (not including the plastic items) for a panel with 10 controls, but it also is easy to design, and customize. There is no need to make a special circuit board for example. This has untold advantages in many areas. It allows very small control panel runs, even in some cases customized for a single user.

The computer 2510 can be used to control a display such as LCD flat panel display 2533 to display information, such as information related to variables set or modified with the controls

The computer 2510 also can optionally take in inputs from other sensors and controls making the system even more versatile. And the processor can inter-relate variables as well. For example a temperature sensor for passenger compartment temperature 2536 can be interfaced to the computer 2510 and used in conjunction with a heater control to affect a desired setting for the temperature. The computer can also provide outputs to human interfaces such as displays or force feedback devices such as vibrators such as 2545 shown, which are used to signal information to someone touching the controls. Similarly the computer can signal via audio signals via a loudspeaker or the like not shown.

The camera can also sense indenting positions of an overlaid member such as plastic member 2540 shown. This allows the member to be used as a touch pad, like on a laptop, or with touch switch positions like many appliances have today. Examples of suitable sensing methods are shown in my co pending applications.

The controls are shown in side view, and other controls such as knobs switches and the like can lie on the surface extending out of the plane of the paper. This is further shown in the technical paper attached.

One may also use a version adapted for the FIG. 25 application including two fixed projectors 2550 (spaced out of the plane of the paper in this view) such as disclosed in the Ser. No. 11/184,076 application referenced above. In this case, the region of the control surface 2530 on which the image data is to be projected needs to be transmissive and dispersive in order to create a rear projection screen, as disclosed in co-pending applications. The added cost is very low (for example 5 dollars), but there is no ability to provide video or real time information unless a separate display is used such as an LCD panel, for example a strip LCD or led type or other flat panel display 2570, in this case requiring an electrical connection to the control surface 2530. Where cost can be afforded one can thus use either an LCD panel or other conventional display or alternatively a low cost LCOS, MEMS, or other video projector such as may be used in cell phone s, remembering too that the drive circuitry for the video display has a cost as well. It is noted that the control surface 2530 may be curved or irregular, without affecting generally the ability of the camera to see the control information desired, such as knob position, finger touch position or the like.

It should be noted that a Fresnel lens such as disclosed in referenced applications and patents may be desirable to direct light from a central light source through all tell tales and labels, but may not be needed for just sensing of the controls, depending on their design. An off axis optical layout can also be used with the camera and light source located for example in location 2538 shown in dotted lines and viewing and illuminating the controls obliquely. Also as shown in referenced applications a mirror may be used if desired to decrease package depth or for other purposes.

FIG. 25b illustrates a one method of providing illuminated labels with an apparatus of FIG. 25a. As shown knob 2570 is mounted on control surface 2510 and is sensed by camera 2515 to determine rotational position, using light from LED 2574, in this case an IR LED at a wavelength which is band passed by filter 2575 in front of the camera, such that light at other wavelengths cannot meaningfully affect the camera image. At night, white LED 2580 (which could alternatively be colored) is turned on, and passes thru a thin slot cut in the member 2560 which has been laser cut with the word VOL (or Volume, for a radio knob). The led 2580 can be controlled to be bright or dim as desired, and within reason numerous other such engraved slots can be cut for other markings. If desired, and if a white LED is used, one can put colored filters on some of the slots in order to make them appear different colors, while also serving to keep smoke or mist out of the housing containing the optical elements. A clear plastic piece can be used for this purpose as well. The band pass filter is used, if needed, to cut out the majority of ambient light passing thru the slots from the passenger compartment.

An advantage of the system is that there are no wires and the complete control panel surface and all its controls can be interchanged easily for another complete design as desired. And because of the optical design having substantial depth of focus, a wide range of panel shapes and curvatures can be accommodated without changing the optical system. For many applications there is no particular need to have a well focused image. For example the target viewed by the camera 2525 on knob 2520 can be somewhat out of focus and yet still its centroid accurately determined by processing the image. Centroid location can then be used to find the knob rotational position as taught in the references.

The optional LCD or other electronic panel display may be completely separated from the control surface, but could also in appropriate cases be mounted to it, if wire connections to the control surface were acceptable. In some applications the control surface may be located below an LCD display. Or it may be above or, less generally, to the side.

The center stack based control panel of many vehicles, such as a 2010 Ford Focus instrument panel center stack appear to be well adopted to the invention herein. A user will change a radio station, for example, using a knob and the station will be displayed on the LCD screen at the top of the center stack. By placing the LCD screen separate from the controls, control surfaces can be easily swapped in and out since no wires connect them.

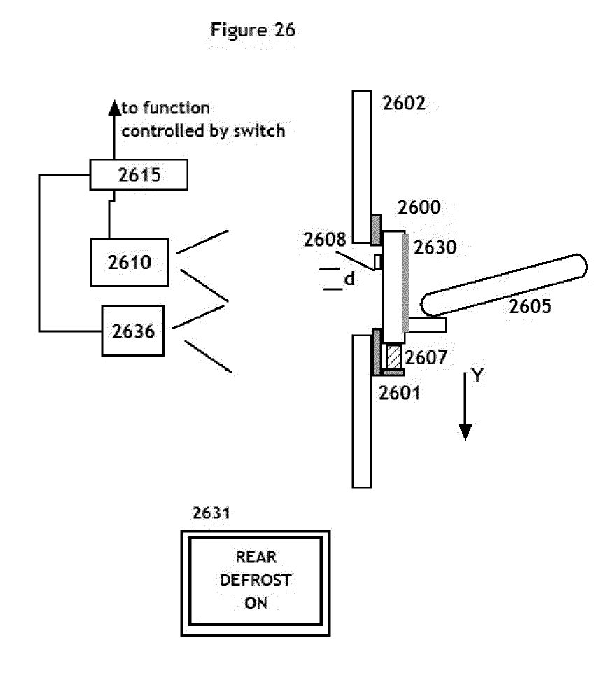

Now disclosed are switches according to the invention which also allow projection of data in their center portions. To make the switch work optically, one can detect a specified movement of the switch (e.g. a one mm movement in the plane of the screen) relative to previous position, or a fixed home position, as disclosed in FIG. 26. Alternatively, one can upon moving the switch expose or cover up a reflector or other light indication which condition is detected, for example by a TV camera. This effectively determines that amount of light above a preset amount is present (or not present) in a given location, indicative of switch condition and is discussed further below. This condition is due to a detected light increase due to the exposure of a reflector to light (or other light source) by the action of moving the switch.

FIG. 26 illustrates an improved switch design sliding substantially in the plane of the screen/control surface whose switching action is accomplished by determining target movement or by exposing or masking one or more detected target datums. As shown, switch 2600 moves in track 2601 in the plane of the screen/control surface 2602 by the action of the users finger 2605 pressing the switch in the Y direction, which is typically in the downward direction in the vehicle, but doesn't have to be. The switch 2601 need only move a small amount “d” in order to allow detect of the movement of target datum 2608 (typically a retro-reflector) on the moving portion of the switch by the camera 2610, (not to scale, and including auxiliary light source if used) with computer 2615 analyzing the image and determining that there is desired a state change of the controlled function, which signal is outputted by the computer to the function in question, such as turning on a light for example. The amount of movement d can be user controlled to be whatever is desired, and multiple values of d for different functions can be used as well. For example for a movement of d1, a certain function can be determined to have been desired by an operator, while for d2 another function. In addition, as noted below the camera system can also determine the movement in two directions (up and down, in this case) with respect to a nominal zero position, if the mechanics of the switch permit such action.

In a preferred version the switch is spring loaded (e.g. by coil spring 2607) to return to its initial position. One historic use of this would be a radio station preset button for example, which you would push in the direction in order to activate that function. Such a push would typically be downward in a car application. But could be in any direction on the plane of the screen and control surface on which the switch moves. An alternative pushbutton into the surface is described in other embodiments below.

As shown the switch 2601, in a preferred embodiment able to reconfiguarably change the switch function label, is provided with a transparent diffusive face member 26260 which can comprise 26M Vikuiti beaded film, a ground surface or other diffusing material. Different types of plastic and arrangements of diffusive surfaces can be used for the switch, and buttons can be solid transparent material or hollow to pass light thru. The screen 2602 itself may have diffusive material such as beaded Vikuiti film on its rear surface (such as 155 in FIG. 1b) but has no diffusion material behind the switch, so that only the face of the switch diffuses the projected radiation from projector 2636 (not to scale).

A front view 2631 is shown of the moving portion of the switch (less finger rest, if any), with the label “REAR DEFROST ON” programmably displayed on its face 2630. At a later time, and for a different function, it might be chosen to display “TRACTION CONTROL ON” for example. The choice of label, its color, and language are all programmable to suit the manufacturer, user or application

The spring member 2607 urges the switch back to its zero position when the user releases his finger. The switch may be constrained by any suitable means for example by housing 2608.

Switch 2601 can also be a multi-position switch, which goes to two or more different positions, besides the zero position (usually an “off” position). The positions are usually in one direction each side of the zero position. In one mode of operation of a three-position switch (center off), for the same radio preset for example, one can push down to select the station and, can push up to set the station for example. This push down is convenient for more used functions.

As an alternative arrangement, one can have three position switch where both positions are used in a time based mode, like window lift, where the up position energizes the window lift motor to raise the window up for the time the switch is engaged, and pushing the switch down similarly moves it down. The same holds true for seat and mirror adjustments.

This is in this form like the slider previously disclosed in the referenced regular applications but in this case it returns to zero when you let off, rather than stays physically at the setting you left it at.

A portion of the housing of the switch may shadow the region around the reflector from incident sun light rays if any. It should be noted that rather than reflect projected light directly one can reroute light thru a switch or knob and cause it to be retransmitted back to the region of the camera so as to be seen thereby as a function of switch or knob position.

The switch just described operates by determining that a displacement has occurred, which displacement is determined to exceed a threshold amount, such as d above in order to determine a switch function.

The necessity to return exactly to a home position, or to exactly go to a contact closure type position to trip a switch such as a conventional switch is not required, though one can build the device to do this. The machine vision system simply can compare a determined starting position of the moving member, to a final position. Neither has to be precise, one only need specify a amount of movement needed (E.g. 1 mm) to trigger a switching action, which movement can be detected by processing the camera image or other means. This movement amount can even be varied for different functions. That is for example, in heater mode, to use a switch like this to select an air distribution condition such as heat on the feet, one might set the amount of movement at 2 mm, where as for use of the same switch in a radio mode as a station preset, it could be set at 1 mm. Or it could be used as a two-position switch for one function, and a three position one for another.

In a variant of the above, one can look for the location in the camera image of the bright spot indicative of a strong reflection caused by exposure of the reflector due to the motion of the switch which positions the reflector in this location. Conversely one can look for the lack of indication in another location. This is particularly useful in a three-position switch as will now be described.

It is noted that best results are obtained when the reflector 2608 can be seen directly by camera 2610. However, it is also possible to see the reflector if the diffusing material such as 26M vikuity is between it and the camera. In this case, and with Vikuiti, a retro-reflective function doesn't work well, and simple reflection may be used.

Next illustrated is a 2 or 3 position switch of the type shown in the above figure in which a reflector is either exposed, or masked by action of the switch, changing the optical signal seen by the camera or other electo-optical sensing device.

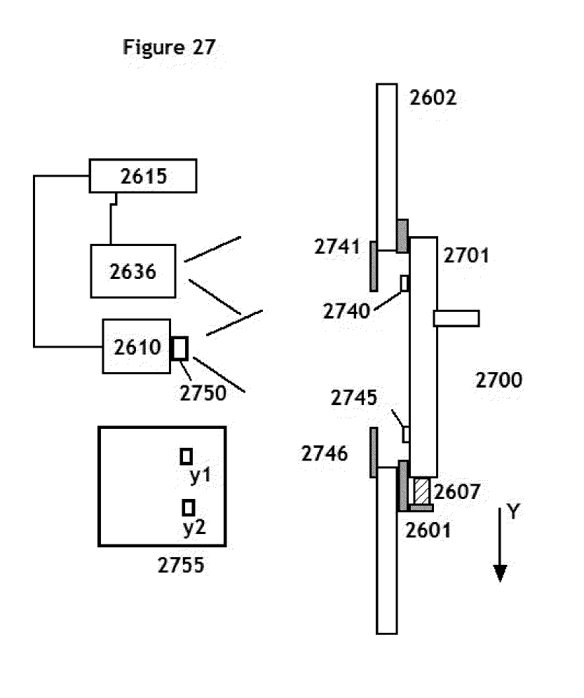

Consider FIG. 27 wherein a reflector is exposed by action of moving the sliding member 2701 so as to direct light to the camera of the invention in order to trip the switch. The apparatus of FIG. 4 can be operated as a three-position switch 2700, using, for example a reflector such as 2740 and 2741 at each end respectively. As you push 2701 down in the drawing from the zero position, 2740 is exposed from behind mask 2741, as you push it up, reflector 2745 is exposed from behind mask 2746, The two images produced by camera lens 2750 appear at different locations y1 and y2 (corresponding to exposing of reflector 2740 or 2745 respectively) on camera image scanning matrix array chip 2755 and thus the presence of a reflected signal at a given location tells whether the push was up or down. No presence, means the switch is at its zero position in this example.

Alternatively one can arrange the switch in an analogous manner where the reflectors are covered up on one side or the other (that is so as to be not exposed to light) due to the movement of the switch. The same sorts of processing can be used, in reverse to identify, which reflector, if any is obscured, and thus which switch direction is intended by the user. In this case the reflector on the side toward where motion is going, is the one to be obscured. At the zero position the both reflectors are seen to be present in this example, opposite to the previously described.

To determine that a switching action has been commanded by the user, the amount of light from the reflector can be compared to a preset value, or to a moving average value within an allowable band, which takes account degradation of certain factors such as light source, dirt on optics etc.

In another mode of operation, an image subtraction can be made in the general camera image, and then the subtracted values compared If this subtraction is performed every fraction of a second or more, the movement of the switch will show up immediately as a positive signal in the otherwise zero intensity subtracted image-assuming no other source of light enters the system, which can however occur in passing through bright sunlight areas.

The two position switch can be one which uses spring steel or another elastic material to give motion that returns to zero, and, if desired, stops against a fixed stop. Different types of springs and materials can be used, such as coil, leaf, or other springs. Alternatively, in those switch embodiments that actually monitor position s, one can utilize a switch, which only approximately returns to its initial position. This can be for example using a springy material such as rubber to hold the switch against the screen and control surface

It is also possible to build a two-axis switch along the lines of the above in which the switch is movable in both the x and y axis by the users finger which movement is opposed by leaf springs or coil springs or other means which urge the switch back to a neutral zero position when finger pressure is removed. The movement in x or y is used to trip the switch as disclosed herein. The two-axis switch can be built to move only in one direction from zero or two directions from zero. Such a three-position switch (including the zero position) is useful in a car context for mirror or seat adjustments in two axes for example.

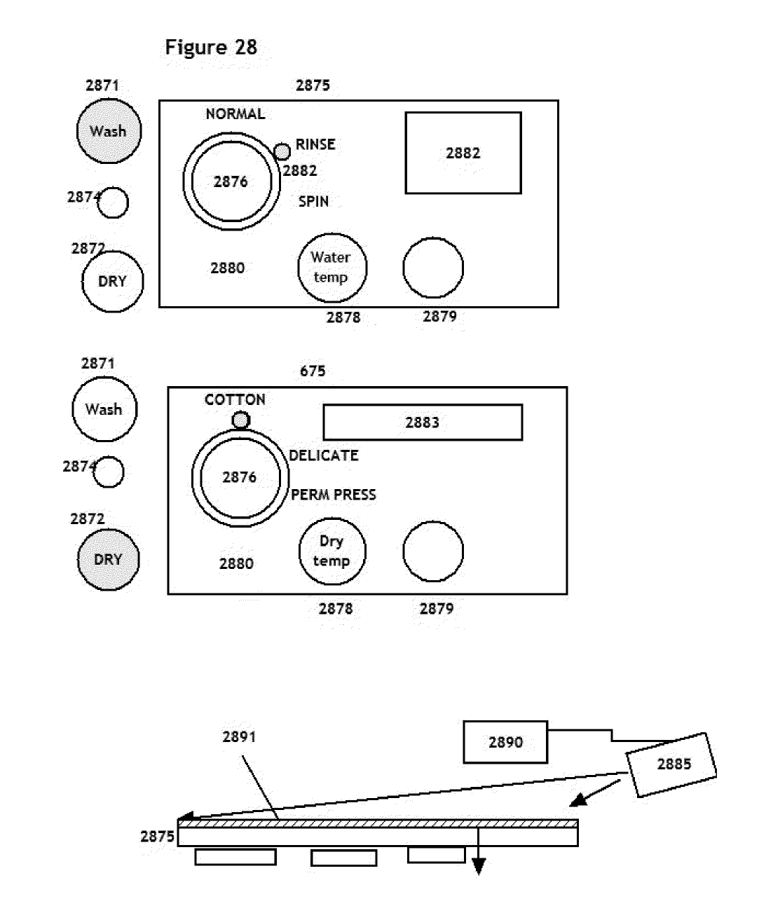

FIG. 28 illustrates a control panel for a combined washer and dryer system for an individual appliance employing a small sensor/projector such as disclosed in my co-pending applications. Projector and sensor combinations of this type for example can be based on the Microvision brand “pico projector”. The projector 2885 is controlled by computer 2890 which receives inputs from the knob, switch and touch screen commands of the system. Video images are projected obliquely onto the rear of the screen and control surface 2875 on which 3M TRAF turning film has been placed to turn the image toward the user. The projected image is over scanned on the screen such that all the screen surface desired may be illuminated.

Two states are shown, each activated by electrical power and control button 2871 or 2872 as desired, to operate a washer or dryer portion of a machine, or pair of machines. Main control and selection knob 2876 which may optionally have a stylish and informative screen in its center as disclosed in my pending Ser. No. 11/045,131 application, and other applications. The knob may be in the form of a ring of a radial thickness t, and if desired (and as disclosed in co-pending applications) may be designed in such a way as to be able to be pressed in or pulled out, to start or stop a cycle, just as many conventional knobs are today. The knob indicator may be a physical pointer, or it may as in 2882 be simply projected on the screen surface, or alternatively it may be projected on the knob face or other knob surface. Two other knobs are provided in this one example 2878 and 2879. More knobs or switches or sliders or other controls may be provided as shown in co-pending applications. These controls may be optically sensed, but they may alternatively or in addition be sensed using electronic means known in the art. One of several desirable versions of the latter is a capacitive touch switch.

A region of virtual touch inputs which may be optionally provided as disclosed in co-pending applications is shown as 2882 for the washer mode and 2883 in dryer mode. These functions or indicators as desired can be different for different modes. Note that when it is desired to connect to another service other than the washer or dryer such as an external program source to listen to watch TV, the optional button 2874 can be pressed, which turns the knob 28728 into a auxiliary selection knob, and other images presented while all the label and other washer function indications can be deleted, or their letters or indications made smaller or otherwise less visible. The washing or drying action can proceed if engaged, as desired.

As noted in my co-pending applications, the image of the TV can be projected to appear right on the screen or on the knob (or switch or other control). This in practice I have found is generally not overly disruptive to the overall understanding of the image, particularly if the knob rings are small in radial thickness so as to not obscure the image substantially.

As shown the projector 2885 (controlled by computer 2890 which receives inputs from the knob, switch and touch screen commands) may be angled to project obliquely onto the rear surface of the screen and control surface at an angle such as shown in co-pending applications. In the case shown, generally desirable with matrix type image chips, the chip may be tilted such that the projected image plane is in focus on the screen from one end to another. If this technique is used, the image may be corrected in the computer so as to present a uniform magnification across the screen. The screen is over scanned at the far end from the projector such that the near end can be fully illuminated.

This system allows one to use projected or otherwise displayed graphics to differentiate appliances, and as pointed out in earlier cases, one can change the control panel and screen shape with ease as well, including the use of curved shapes and cut out peripheries. One can easily change language to suit different markets or buyers. And one can display patterns, colors and the like also on a knob or other control element face as pointed out in my co-pending applications.

Disclosed in other co-pending applications is an embodiment of the invention serving as a control panel and dashboard for a Combine, Road Grader, or other working machine, including a military machine. In this case, rather than combine the two functions on a single panel, an alternative (for this and other embodiments) may be constructed in which a slid-ably interchangeable control panel is employed to switch function from a vehicular movement, to working machine. The advantage is the directly in front of the operator is all the controls and data he needs for the task at hand. There are other ways of interchanging the panels, such as physically taking one off and putting one on, using a turret arrangement or whatever.

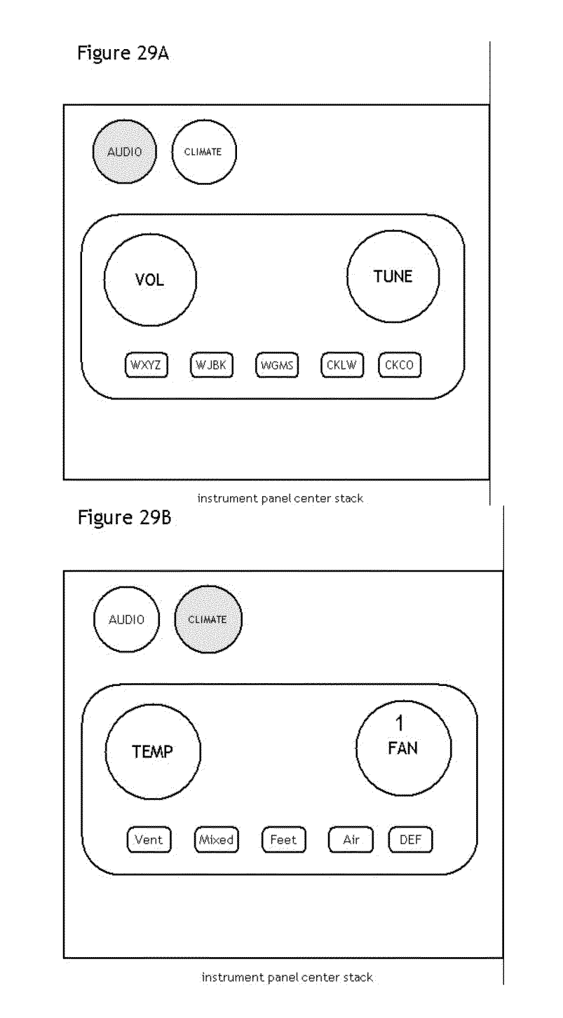

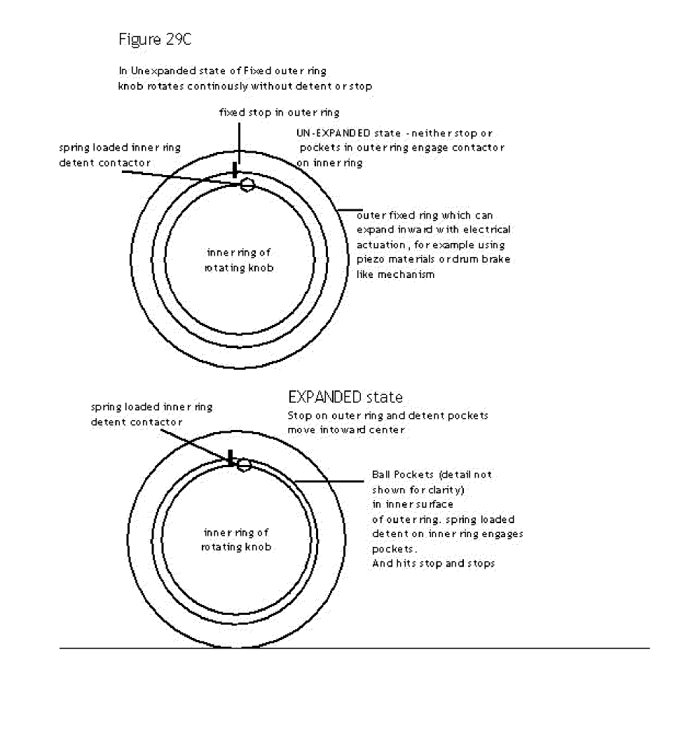

FIG. 29 illustrates a version of the invention for the center stack of the vehicle further in the Radio-Heater metaphor mentioned in my referenced co pending applications. FIG. 29A illustrates the unit in audio system mode, achieved by touching the “Audio” button, which causes the video display to label the controls accordingly. FIG. 29B illustrates the unit in Climate control system mode, achieved by touching the “Climate” button, which serves to change the projected image data, and the control outputs of the control system used. The point I want to make here, is that the user can easily learn the location of the basic physical control details, in this case two knobs 2905 (the Left knob) and 2906 (the Right knob) and 5 switches 2910-2914, since the controls are in the same place for both the Audio and heat functions. The person only need remember which mode he is in, Audio or Climate, and this can be reminded by having the appropriate light 2901 or 2902 lit up, for example. And words such as “AUDIO” also displayed in big letters in the language of his choice on the screen and control surface 2900.

Thus an argument is that this is more intuitive than today's almost universal arrangement of having both Audio and Climate as separate modules, but with absolutely no common features between them. This is even more the case, since to do this occupies space, which means that to squeeze these separated modules into the same space, one has to make the individual controls, and their labels, smaller and more difficult to work and see. For many older drives in fact, it is not possible to safety discern the labels on many such devices while driving.

I believe that what I have invented is a significant improvement over the basic car of today, leading to more enjoyment and less driver distraction. It is now of interest to point out that FIGS. 29 A and B are the most common layouts of my controls which would preserve the traditional layout. Why? Because the audio system has for years had a volume control, a tuning knob, and 5 or so preset buttons for favorite stations. I have just carried this layout over to the Climate control, which the knobs for temperature and fan speed, and buttons for the vent air distribution. These functions are themselves at least somewhat standard too, so the whole arrangement departs little from what people, particularly (but not necessarily) older people, are familiar with-except that each module, is only workable by being in that mode.

In so doing the above, it is interesting to note that both Volume and Temperature are historically analog continuous functions. Thus the same Left knob with no detents or just little closely space “notch” detents can suffice for volume and temperature functions. I have shown this knob purposely on the left as it closest to the driver, as it is my belief that volume in the audio case, and temperature in the climate case, are the most important, or at least most used, functions.

In the case of the Right knob, in the Audio mode, the tuning knob is continuous. In Climate mode, the fan speed can also be continuous using a rheostat, even though most fan units in cars today are operated with a fixed set of speeds (usually 4 or 5). Or the speed is selected automatically in some cars with “automatic climate control”. In the Right knob case, if one wants to have it like today, then one can have a continuous knob when in Audio mode, with a program-ably actuated detent when in climate mode. I have disclosed such a device in co-pending applications, but will further point out a simple version below in FIG. 29C.

The switches 2910-2914 operate tactilely the same for both presets and air distribution. Push one in (or slide it, if that type used) and the station changes to that station, or the air comes out that vent set.

One can have all manner of optional control functions with my invention, achieved with touch screen type functions, or added physical controls or a combination of the two. For example, touching a touch icon could make the preset buttons correspond to a different set of stations, perhaps on a different band (e.g., FM vs. AM). The important thing though, is that the basic functions are standard and easily committed to memory and easily found and controlled while driving. This is a major advance re Driver Distraction. Also important is that when desired, the Audio and climate functions may themselves be replaced by something else, such as a play list or a big image of a map or a rear camera picture for example.

It should be noted that pictographs or abbreviations can be substituted for words, if desired or for size reasons. However the ability of the invention to change the language of the label makes hard to understand pictographs less necessary.

Previous disclosures for example Ser. No. 12/715,097 in FIG. 16, have illustrated a simple detent mechanism that could be used for the right knob above. When Climate mode is turned on, a member is actuated by electromagnetic or piezoelectric or other known means to ride on the outer diameter of an inner ring of the knob as the knob is turned by the user, the actuated lever contacts the detents (e.g. 5) on the ring representing the 5 speeds of the knob. The momentarily hesitation resulting signals the person turning the knob accordingly, as he for example ramps up speed 1-2-3-4-5. In this case the speed “1” is projected into the middle of the knob, though it could be around it, and laid out conventionally like a clock.

This Right knob in this case however has one difference. Rather than go to a fixed location where it can go no further, which takes added electromechanical complexity, this knob can be turned further, in which case the speed sequence starts over, or if desired, goes back the other way. For example 5-4-3-2-1. One company, Immersion corporation, builds knobs with much more versatile detent combinations than 1 have just disclosed, however, they to my knowledge do not have one that leaves the center of the knob clear for projected information as this does, nor one as low cost. It is however possible to use one or more immersion knobs with my invention, if labels are projected next to, rather than in the middle of, the knob.

Alternatively, FIG. 29C illustrates an alternative knob with a clear center that duplicates today's fan speed operation knobs. It has a programmably actuated outer ring providing detent pockets and a fixed stop when actuated. The tops can be provided at both ends, rather than just one as shown, in order that operation be similar to a discreet fan speed knob in common use today.

Referring now to other embodiments, one may also provide a flip up and over tray table like version in an airplane seat behind a bulkhead, or in a wheel chair or other chair, in which the projector is on the seat between the legs of the user.

It should be noted that the embodiment of FIG. 5 in the curved portion of an instrument panel in front of the passenger, may also be thought of in the curved arm of an arm chair, where a person seated in the chair may operate computer systems by touching a screen/control surface in the curved arm, or by actuating a physical control on the surface. Such a system might be a remote control for a TV for example.

The invention herein applies for the control of anything where the projection and sensing element is desirably separated from the user interface element of the invention. This can include controls outside clean rooms, nuclear facilities and the like, with windows or free space separating the two elements.

FMVSS 101 is contained in USA CFR Title 49 part 571. “Light” as used herein includes all electromagnetic wavelengths from ultraviolet to near infrared.

With respect to figure is 29 it should be noted that the choice between one or the other states of the display and controls (for example audio or climate as shown) can be made using buttons or other controls on the steering wheel, which are quite convenient for the driver. It is contemplated that the buttons themselves, or other buttons on the steering wheel indeed could also reconfigure their functions, depending for example on the choice of whether the audio or the climate selection was made. For example, if the driver press climate as we have shown it, substantially the whole center stack screen and display can if desired be programmed to display the various controls for the heating and air conditioning, or any other choice, programmed - - - either at the factory or by the user himself. In any case it would be useful than that buttons on the steering wheel that might of at one point been used to change volume or station with respect to the a radio portion of the vehicle would in the climate control mode change their function to for example temperature and fan speed. By having these buttons reconfigure, fewer are needed on the steering wheel and the resulting steering wheel buttons can be larger and less confusing to the driver.

It is also possible that one might choose to have the buttons on the steering wheel operate in an opposite manner from the state of the center stack display and controls. For example, if one used a button on the center stack such as shown in FIG. 29 to change let us say the state of the center stack controls to the climate condition, one might purposely leave some steering wheel controls in the audio mode so that you would then have steering wheel controls to keep operating the basic functions of your audio system while you use the center stack in detail to set up your climate system, which of course you would be able to do given the large amount of data that can be manipulated and presented on the center stack using the invention. It is possible that the operator can be given the choice of whether what sorts of ways of operating the system he prefers pointed out in other applications.

It is possible to use a DLP or other 2-axis light valve type projector to do the sensing task as well. This typically provides a lower cost solution and one which shares a common optical path I have found an improvement to what I've described previously can be made in this context using the projector to also function as a center where speed can be attained sufficient to both project and sense without the loss of image quality provides. In this example, it is useful to modulate the light source, either the visible LEDs or diode lasers of the projector or an auxiliary infrared LED or diode laser, in order to achieve a discrimination against ambient light using time based or frequency based detection techniques. In this particular case, the image elements of the image engine DLP or LCD or LCOS for example provide the ability to determine where on the screen and control surface the information is coming from and therefore locate a touch on the screen or a control position such as a rotational location of the knob.

I also found another a good way to change the state of the control surface in screen (for example in FIG. 29 from audio system to climate control system) is to have one of the knobs preferably the one closest to the driver simply also function as a push switch. For example, if you're in a temperature in an a climate control mode and your hand is on the temperature knob, which is closest to you and you push it in that can very conveniently cause the system to change to audio system in which case the knob now represents volume which is also the typical thing you would seek to change in an audio mode. In other words, you can do it all in one motion from the thing most changed in climate to the thing most changed in audio the volume, changing the rest of the control surface and screen at the same time.

In the rear seat display screen situation of FIG. 5b for example it is possible to have a screen such as 545 instead located in the headrest of the seat in front of the rear seat passenger, rather than in the front seat itself. A video projector located in the seat proper illuminates the screen, and data inputs of the passenger on the screen or control associated with it are optionally sensed according to the invention. Such data can be related to internet addresses if the unit is used for searching, commands to change entertainment information such as DVDs or video clips, or the like.

This design is also conducive to safety in the event of an accident. The headrest for example can move forward in the fore aft direction of the vehicle (perhaps using a pyrotechnic charge) to better support the person's head, while the projector, sensor and electronic components remain in the seat proper, the head rest moving generally perpendicular to the optical path of the projector and sensor (if an optically based touch sensor is used). Thus only plastic parts need be contained in the headrest itself, and these may be as soft as desired for safety purposes. This includes not only the screen and control surface, but any knobs or push switches that might be attached thereto.

The invention has been described in connection with numerous embodiments, it is to be understood that the specific mechanisms and techniques that have been described are merely illustrative of the principles of the invention, and numerous modifications may be made to the methods and apparatus described without departing from the spirit and scope of the invention.

In the above figures, detents of the classical mechanical kind can be built into a knob or slider and it's mounting. In addition, a computer may also control vibrator wave source (such as used for example in Cell phones or pagers) to excite the screen and any physical details such as knobs or switches thereon to provide a “programmable” feel in conjunction with any visual data presented. This can give a different feeling or vibration for each position of a knob, for example. And one can have different frequencies than the other, so as to be distinguishable one from the other. The actual location of the settings can be determined by the point at which the feel is felt, as a programmable position detent, so to speak. In this case the display may be varied as well. The sensed indication of the knob datum in one of the rotational positions is used to provide input to control the programmable wave source. For example if the volume knob is most counter clockwise, the volume could be low, and the vibration amplitude low as well, when the knob was fully clockwise to its max setting, the vibration amplitude could be highest.

Lettering, Labeling, and Knob (Or Other Physical Control) Size. For most automotive control applications studies by transportation researchers suggest that lettering for controls in the center stack more or less in the drivers line of sight, should be at least 5 mm.times.5 mm per letter, and larger perhaps for elderly drivers and others who could benefit by it. For a high resolution density of 5.times.5 pixels per character for example, 5 mm high letters would only require 250.times.250 pixels to be projected for a 250.times.250 mm (10.times.10 inch) display. This is easily achieved with even modest projector or other display technology today. Of course, higher resolution allows more dense displays, for various data and other images that could be desirable. Resolution of 1280.times.1024 pixels is commonly available if required (albeit at more cost).

I believe that lettering 7 mm high or more is very desirable for ease of reading while driving. If the character size is for a simple example 7.times.7 mm, then a center stack of 250 mm (10 inches approx) width can accommodate 250/7 or approx 34 letters and spaces of equal size. If two large horizontally aligned knobs of 50 mm are used, which are big, and easy to grasp, this reduces the amount of lettering space to a minimum 50 mm in the direction horizontally through the knobs, or approximately 22 letters, if one goes right to the edge of the center stack with the projected data (an advantage of the invention in its projection embodiment). This is still sufficient for most purposes, either to show data relating to the knobs, or to add information in the space between the knobs. But only with a large screen substantially running right to the edge of the available space in the center stack (or elsewhere) can one achieve having such large letters, and such big knobs.

Knobs and switches are today the most common and accepted physical control means for automobile instrument panel use. It is my opinion that knobs of at least 25 mm in diameter are desirable for this application, and preferably 35 mm and above for use by older people or those with disabilities. And people in general, who wish to grab a knob without looking too hard where it is. Larger knobs such as those in the 35-50 mm diameter range typically allow more selection positions and may also more easily incorporate clear centers through which screen data concerning the knob action can be projected and viewed. Or as pointed out elsewhere the knob front surface itself may constitute the screen, which diverges light to the driver representing data projected on to it. The invention thus comprehends projection of data through the center of knobs, sliders or switches, aiding comprehension

Where smaller knobs such as 25 mm are used, one may of course put more of them on a given screen/control surface, than if knobs twice the size are used. Again the availability of larger lettering space for these knobs is desirable. Realizing too, that the lettering size can be varied by the computer controlling the display in order to suit the needs of the driver. And in special high stress situations it might be automatically increased in size (or conversely, temporarily eliminated, in order to make room for important other information such as road hazard warnings or video images of consequence.

Preferably, the screen/control surface of the embodiments above, and other automotive instrument panel embodiments herein is large, on the order of 9.times.7 inches or larger, to provide the advantages of larger physical controls (e.g. knobs) and lettering, plus a substantial display area for information and video images, as well as a surface for virtual controls as needed (or optionally, added physical control details). In my opinion it should be as large as possible with in vehicle in question, and it is not unreasonable to consider even 12.times.14 inch or 10.times.18 inch RTD control displays. And some vehicles could even employ more than one such RTD device within a given instrument panel complex. For example, they could be in an over-under arrangement, or one on the right side of the steering wheel with another on the left.

The position or movement of any knobs (rotation) or levers/switches (linear motion) is monitored, as is the location of finger touch on the screen (where virtual controls are desired). In one preferred embodiment, the same machine vision system performs both functions, and is integrated with the computer control of the display and the force feedback. The total system is elegantly simple, and allows for a myriad of additional features. Particularly of interest are those in which the tactile aspects of the instrument panel can be tailored in their entirety to the needs of individual users or the desires of individual vehicle model development.’

As pointed out elsewhere and in co-pending cases it may be useful to subtract the background with or without the illumination source on from the instant data. Because this adds cycle time and some transient background radiation may exist, a higher frame rate than 30 frames per second typical of TV cameras is desirable for best results. This can be achieved locally with ease (e.g. in the region of a knob) using a pixel addressing CMOS camera for example, which can easily provide data to the computer to allow it to do several hundred alternate background subtracts per second. One generally only need scan the region such as an annular ring on the back of the knob where the targets travel around for example, or a linear strip for the position of a slider datum, or a projected icon location where touch is desired (noting that unlike the physical devices whose general positions are fixed, these touch icons can be varied programmably in their location, in which case the scan region of the camera needs to be varied to match.

The knob targets are typically bright on a black background, but they could be reversed. In this way (dark on bright background) they would not be confused by stray light brightness zones caused by large sunlight loads, for example three targets would be black on a white or silver annular knob background, which could in some cases if desired be a retro reflective background for added contrast.

The invention has been described in connection with numerous embodiments, it is to be understood that the specific mechanisms and techniques that have been described are merely illustrative of the principles of the invention, and numerous modifications may be made to the methods and apparatus described without departing from the spirit and scope of the invention.

1. A method for providing a sterile control interface responsive to a user input, comprising the steps of:
providing a control member including at least one physical control for operation by a user thereof;
sterilizing said control member;
providing an electro-optical sensor to view said physical control, said sensor having an output;
providing a computer; and
using said computer to analyze said sensor output, determining a position of said physical control indicative of a user input.

2. A method according to claim 1 including the further step of projecting information relating to said input.

3. A method according to claim 1 wherein said sensor views said physical control from the rear thereof.

4. A method according to claim 1 wherein said sensor views said physical control from the front thereof.

5. A method according to claim 1 wherein said physical control is a knob whose rotational position is determined.

6. A method according to claim 4 wherein knob position or state in at least one additional axis is determined.

7. A method according to claim 1 wherein said electro-optical sensor is a digital camera.

8. A self-sterilizing control panel comprising:
a control member having a surface, said member having a front side on which a control input is made by a user;
an electro-optical sensor to sense position of said control input, said sensor located behind said control member and producing an output;
a sterilizing radiation source located behind said control member to irradiate said control surface; and
a computer to control said radiation source and said sensor and to output a control signal based on said sensed position.

9. Apparatus according to claim 8 further including a physical control device.

10. Apparatus according to claim 8 wherein said control input is finger touch position or movement.

11. Apparatus according to claim 8 wherein said control input is knob rotation.

12. Apparatus according to claim 8 wherein said sterilizing radiation is ultraviolet light.

13. Apparatus according to claim 12 wherein said ultraviolet light illuminates the user contact surface of a physical control device.

14. Apparatus according to claim 8 wherein said control member is movable to an irradiation location.

15. Apparatus according to claim 8 further including a video projector to project image information on said control surface from the rear thereof.

16. Apparatus according to claim 8 wherein said sensor is a digital camera.

17. A device for sensing positions or movements of persons or objects having reflectors provided thereon, comprising:
a computing device having a camera and at least one LED light source to illuminate objects in the camera field of view;
a band pass filter attached to said device to filter light returning to said camera; and
software to determine from said camera the image positions or movements of said objects.

18. Apparatus according to claim 17 wherein said computing device is a smartphone.

19. Apparatus according to claim 17 wherein said reflector is a retro-reflector.

20. Apparatus according to claim 17 wherein light source is a white light source, and said band pass filter filters both light from said source and said returning light to said camera.
